(19) United States
(12) Patent Application Publication
(10) Pub. No.: US 2015/0004620 A1
(43) Pub. Date: Jan. 1, 2015

(54) METHOD OR DEVICE FOR IDENTIFYING INHIBITOR OF EPITHELIAL MESENCHYMAL TRANSITION

(71) Applicant: Agency for Science, Technology and Research, Singapore (SG)

(72) Inventors: Jean Paul Thiery, Singapore (SG); Kian Nigiap Chuo, Singapore (SG); Wen Jing Sim, Singapore (SG)

(21) Appl. No.: 14/369,549

(22) PCT Filed: Dec. 28, 2012
(86) PCT No.: PCT/SG2012/000491

§ 371 Date: Jun. 27, 2014

Related U.S. Application Data
(60) Provisional application No. 61/582,276, filed on Dec. 31, 2011.

(51) Int. Cl. G01N 33/50; G01N 33/574

(52) U.S. Cl. CPC G01N 33/5011; G01N 33/574; G01N 33/5044; G01N 2500/10
USPC 435/6.13; 435/7.1; 435/288.7; 435/287.2; 435/29; 435/288.4; 702/27

(57) ABSTRACT

A method of identifying inhibitors of epithelial-mesenchymal transition (EMT). The method may comprise comparing different sets of image data obtained from one or more cell colonies before (T1) and after (T2) exposure to a possible inhibitor of epithelial-mesenchymal transition. The method may further comprise measuring the cell number and a spreading coefficient value in the one or more cell colonies for determining cell count ratio (CCR) and normalized cell dispersion ratio (CDR) for the one or more colonies. The possible inhibitor may then be identified to be an inhibitor of EMT if the determined CCR and CDR indicates that the possible inhibitor i) does not or marginally inhibit growth and inhibits EMT, or ii) inhibits growth and inhibits cell dispersion and optionally inhibits also EMT, or iii) is cytotoxic and inhibits cell dispersion and optionally inhibits also EMT.

### CROSS-REFERENCE TO RELATED APPLICATIONS

This application claims the benefit of priority of U.S. provisional application No. 61/582,276, filed 31 Dec. 2011, the contents of it being hereby incorporated by reference in its entirety for all purposes.

### TECHNICAL FIELD

The present invention generally relates to the field of drug screening. More particularly, the present invention relates to methods, devices or system for screening and identification of compounds that inhibit epithelial-mesenchymal transition in a proliferative disease such as cancer.

### BACKGROUND

Epithelial Mesenchymal Transition (EMT) is a crucial mechanism for carcinoma progression, as it provides routes for in situ carcinoma cells to dissociate and become motile, leading to localized invasion and metastatic spread. Targeting EMT therefore represents an important therapeutic strategy for cancer treatment. The discovery of oncogene addiction in sustaining tumor growth has led to the development of modern molecular targeted therapeutics. These small molecule inhibitors function by binding to the ATP-binding site of the dysregulated kinase oncogene, thereby inhibiting the phosphorylation and activation of its signal transduction cascade responsible for sustaining tumor growth.

Many preclinical studies have showed the effectiveness of targeted small molecule inhibitors in killing cancer cells or preventing tumor growth. Examples include Imatinib Mesylate for the treatment of chronic myeloid leukemia and Gefitinib for the treatment of non-small-cell lung cancer. Whilst originally identified and optimized for their anti-proliferative effects, evidence suggests that some of these targeted small molecule inhibitors may also inhibit EMT initiation or sustenance, since the EMT program is modulated by similar signaling pathways for which these molecules have been generated. For example, Ki26894, an ALK5 inhibitor, has recently been shown to decrease the invasiveness and EMT of scirrhous gastric cancer cells. However, an extensive screening effort to identify and quantify the relative effectiveness of existing targeted small molecule inhibitors in EMT modulation has not been methodically attempted. Thus, there is a need to provide a screening assay that can identify the EMT modulating properties of compounds.

### SUMMARY OF INVENTION

According to a first aspect, there is provided a method of identifying inhibitors of epithelial-mesenchymal transition (EMT). The method can comprise comparing different sets of image data obtained from one or more cell colonies before (T1) and after (T2) exposure to a possible inhibitor of epithelial-mesenchymal transition. The method can further comprise

measuring the cell number and a spreading coefficient value in the one or more cell colonies for determining cell count ratio (CCR) and normalized cell dispersion ratio (CDR) for the one or more colonies. The possible inhibitor can then be identified to be an inhibitor of EMT if the determined CCR and CDR indicates that the possible inhibitor i) does not or marginally inhibit growth and inhibits EMT, or ii) inhibits growth and inhibits cell dispersion and optionally inhibits also EMT, or iii) is cytotoxic and inhibits cell dispersion and optionally inhibits also EMT.

In a second aspect, there is provided a device using the method as described herein for identifying inhibitors of epithelial-mesenchymal transition.

In a third aspect, there is provided a system comprising a device as described herein and a camera for recording an EMT time-lapse video.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a schematic of the spot migration screening assay to identify EMT inhibitory compounds. EMT can be initiated and maintained in epithelial cells via growth factor signaling. This assay measures the dispersion of cells in the presence of a test compound and an EMT inducer (EGF, HGF or IGF-1). The prevention of cell dispersion directly correlates to the propensity of a test compound to block an induced EMT signaling pathway. B, screening assay image acquisition workflow. Robot-assisted plating of H2R-mcherry transected NBT-II cells into the well centers of 96-well plates. The initial plate image acquired at T1 served as the baseline reference for calculating the CCR and CDR values for each well. The cells were treated with test compounds overnight and further incubated for 24 hours with a growth factor to induce EMT. C. final plate image acquired at T2 depicted the dispersion response of cells 24 hours after addition of the compounds and growth factor treatment. In the example shown, columns 2-11 were treated with 80 different test compounds at 6.67 μM and EGF. Column-1 served as negative controls treated with 0.67% DMSO and EGF, while column-12 served as positive controls treated with 6.67 μM AG 1478 and EGF. D. magnified images of selected wells acquired at T1 and T2. Wells C12, H03 and D02 are examples of cell colonies treated by compounds that inhibited EGF-initiated cell dispersion and did not inhibit cell growth. Well C01 is a cell colony undergoing EGF-induced EMT without any dispersion inhibition. Well E09 is a cell colony treated by a growth inhibitory or toxic compound. Thus, FIG. 1 provides an overview on how the method of screening and identifying inhibitors of EMT may be implemented.

FIG. 2 is the image processing procedure to determine the cell count and dispersion values of a well. A. colony nuclei image of each well was obtained by stitching four adjacent, non overlapping fields together. The example here shows a primary cell colony surrounded by several cell outliers, B, nuclei segmentation, which consists of a wavelet transform and watershed algorithm steps, was applied to identify all nuclei in the well. C. the nuclei segmentation mask was then dilated to generate merging region areas where distinct cell clusters could be isolated. In general, a primary large region, representing the cell colony of interest, and other smaller regions, representing outlier cell clusters, was identified. D. nuclei within the colony of interest were kept for measurement. Cell count was determined by the total nuclei count within the colony. Cell dispersion was determined by applying the spreading coefficient formula. The arrow shown represents a vector centered on the colony center with distance equal to the spreading coefficient. Thus, FIG. 2 illustrates how total cell count and cell dispersion rate as used in the method of the present invention may be determined from collected image data.

FIG. 3 is a dot plot illustrating the cell dispersion ratio (CDR) vs. cell count ratio (CCR) of the behavior of NBT-II cells treated with different test compounds and growth factors in a spot migration assay. CDR threshold was set at 50% CDR between positive controls' average CDR and negative controls' average CDR. CCR threshold was set at 1.5 growth rate, which corresponds to normal growth rate of NBT-II cells during the T1-T2 time period, in the absence of an EMT inducer. Compounds assessed are compounds that inhibit cell dispersion (i.e. less than CDR threshold) and do not severely inhibit cell growth (i.e. more than CCR threshold). To further refine hits, the test compounds were run at a low and high concentration (1.67 and 6.67 μM. respectively). Hit compounds (crossed squares) were classified as test compounds that satisfy the CDR and CCR threshold criteria at both concentrations. Thus, FIG. 3 provides an example of how a compound of interest may be assessed using the method as disclosed herein for its ability to inhibit cell dispersion whilst do not severely inhibit cell growth.

FIG. 4 is a collective T2 plate images and CDR dose response profiles of EGFR inhibitor, Gefitinib, against EGF-(A), HGF-(B), and IGF-1-(C) induced EMT. FIG. 4 demonstrates that the method as described herein can detect compounds that are effective against EMT by specific growth factors.

FIG. 5 is a collective T2 plate images and CDR dose response profiles of ALK5 inhibitor. A83-01, against EGF-(A), HGF-(B), and IGF-1-(C) induced EMT. FIG. 5 demonstrates that the method as described herein can be used to detect compounds that are potent against EMT with EGF, HGF or IGF-1.

FIG. 6 is a graph illustrating the robustness of spot migration assay. Cell Dispersion Ratio of positive controls [6.67 μM AG1478 (A), 6.67 μM JNJ-38877605 (B), and 6.67 μM BMS-536924 (C)] and negative controls [0.67% DMSO] (A-C) for three experiment sets of triplicate plates (i.e. 8 data points per control condition per plate) is shown here for the spot migration assay against EGF (A), HGF (B). and IGF-1 (C) induction, respectively. Thus, FIG. 6 demonstrates the consistency of the cell dispersion ratio of positive controls and negative controls.

FIG. 7 illustrates the modulation of EMT markers, E-cadherin and MMP-13, by EMT inhibitors under EGF-(A), HGF-(B). and IGF-1-(C) induced EMT conditions. E-cadherin expression levels decreased with growth factor addition compared, with DMSO control, indicating cells have undergone EMT. However, the E-cadherin level was restored or augmented with increasing compound concentrations, due to EMT inhibition effected by these compounds. Conversely, addition of HGF and IGF-1 increased MMP-13 expression levels compared with DMSO control. In general, this increase in MMP-13 expression could be abrogated with the addition of the EMT inhibitors. Positive control for each panel: 2 μM AG1478 (A), JNJ-38877605 (B), and BMS-536924 (C). Thus, FIG. 7 provides an example of possible downstream analysis that may be conducted subsequent to the detection of possible inhibitor by the method as described herein.

FIG. 8 is a phase contrast image of A549, Calu-1 and H165.0 cell lines after AZD0530 treatment demonstrating the compaction of cell morphology in lung carcinoma cell lines induced by AZD0530. Cell lines were treated with 2 μM AZD0530 or 0.02% DMSO control for 48 hours. Scale bar is 100 μm. Thus, FIG. 8 provides an example of how the method as described herein may be validated by the use of various lung carcinoma cell lines.

FIG. 9 is a phase contrast image of A2780J, DOV13 and HEY cell lines demonstrating the compaction of cell morphology in ovarian carcinoma cell lines induced by AZD0530. Cell lines were treated with 2 μM AZD0530 or 0.02% DMSO control for 48 hours. Scale bar is 200 μm. Thus, FIG. 9 provides an example of how the method as described herein may be validated by the use of various ovarian carcinoma cell lines.

FIG. 10 is a western blot image showing the increase in epithelial marker plakoglobin expression induced by AZD0530 in cell lines. Cell lines were treated with 2 μM AZD0530 or 0.02% DMSO control for 48 hours. Thus, FIG. 10 provides an example of possible downstream analysis that may be conducted subsequent to the detection of possible inhibitor by the method as described herein. In this figure, western blot analysis was used to validate the possible inhibitor detected by the method as described herein.

FIG. 11 is a western blot image showing the increase in epithelial marker E-cadherin expression and the decrease of mesenchymal marker MMP-13 expression in panel of ovarian carcinoma cell lines treated with AZD0530. Cell lines were treated with 2 μM AZD0530 or 0.02% DMSO control for 48 hours. Thus, FIG. 11 provides an example of possible downstream analysis that may be conducted subsequent to the detection of possible inhibitor by the method as described herein. In this figure, western blot analysis was used to validate the possible inhibitor detected by the method as described herein.

FIG. 12 is a bar graph showing the decrease in average velocity of cell migrations in a panel of ovarian carcinoma cell lines treated with AZD0530. Cells were treated with 2 μM AZD0530 or 0.02% DMSO control for 48 hours and tracked for 24 hours. Thus, FIG. 12 provides an example of how the method as described herein may be validated by the use of various ovarian carcinoma cell lines.

FIG. 13 is a bar graph showing the decrease in average displacement cells migrate in a panel of ovarian carcinoma cell lines after treatment with AZD0530. Cell lines were treated with 2 μM AZD0530 or 0.02% DMSO control for 48 hours and tracked for 24 hours. Thus, FIG. 13 provides an example of how the method as described herein may be validated by the use of various ovarian carcinoma cell lines.

FIG. 14 is an x,y plot illustrating the individual tracks of cells, imaged using Metamorph software controlled time-lapse microscopy for 24 hours. Each cell is tracked using the particle tracking routine provided by the Metamorph software, which automatically detect and generate the x and y coordinates of the selected cell being tracked. The x and y coordinates are used to plot the movement of the individual cell. The figure demonstrates suppression of the mobility of ovarian carcinoma cell lines treated with AZD0530. Cells were treated with 2 μM AZD0530 or 0.02% DMSO control for 48 hours and tracked for 24 hours. Thus, FIG. 14 provides an example of how the method as described herein may be validated by the use of various ovarian carcinoma cell lines.

### DEFINITIONS

As used herein, the singular form “a,” “an,” and “the” include plural references unless the context clearly dictates otherwise. For example, the term “a gene” includes a plurality of genes.

### DETAILED DESCRIPTION OF THE INVENTION

Epithelial Mesenchymal Transition (EMT) is a fundamental process driving embryonic development particularly during gastrulation and in morphogenesis of the heart primordium, neural crest and somites. Cells engaged in the EMT process undergo complex changes in cell architecture and behavior. In a typical epithelial layer, epithelial cells develop adhesive structures between adjacent cells, such as adherens junctions, desmosomes and tight junctions, to establish robust intercellular adhesions. Epithelial cells are apico-basal polarized, with the apical and basal surfaces serving different functions. Mesenchymal cells, on the other hand, do not have stable intercellular junctions and possess front-to-back leading edge polarity. These characteristics also increase the migratory capacity in mesenchymal cells, owing to the shift of weaker cell-cell adhesion and stronger cell-matrix adhesion. Thus, the EMT process describes a series of events during which epithelial cells lose many of their epithelial characteristics and take on properties that are typical of mesenchymal cells.

For more than a decade, EMT has been recognized as a potential mechanism for the progression of carcinoma. At the onset of tumor progression, dysregulation of the cell cycle machinery can result in proliferation of the normal epithelia to give rise to an adenoma, the adenoma, with additional genetic and epigenetic alterations, can later progress to a carcinoma in situ. The carcinoma in situ is believed to engage the EMT program at the micro-invasive stage, allowing individual carcinoma cells to migrate and intravasate into lymph and blood vessels and eventually disseminate and metastasize to distant organs.

Metastasis of the primary tumor is assisted by the release of cytokines and growth factors that are secreted by die surrounding stroma. Cancer patients are reported to have elevated serum levels of growth factors such as but not limited to hepatocyte growth factor (HGF), epidermal growth factor (EGF), transforming growth factor-beta (TGF-β), insulin-like growth factor-1 (IGF-1) and the like. In addition, numerous carcinoma are found to have over-expression of either wild-type or mutated kinases. These kinase oncogenes play important roles in growth factor signal transduction regulation, and their dysregulation can lead to survival and excessive proliferation of cancer cells as well as the initiation and sustenance of the EMT program and tumor metastasis. These findings have generated great interest in understanding the role of oncogenes and their signaling cascades in tumor growth and the EMT program.

The discovery of oncogene addiction in sustaining tumor growth has led to the rapid development of targeted therapeutics. Whilst initially optimized as anti-proliferative agents, it is likely that some of these compounds may inhibit EMT initiation or sustenance, since EMT is also modulated by similar signaling pathways that these compounds were designed to target. Thus, there is a need to provide a method that can extensively screen small molecule inhibitors to identify and quantify their effectiveness in modulating EMT.

Accordingly, the present disclosure relates to the design and development of epithelial-mesenchymal transition (EMT) inhibition drug screening assay. In particular, the present invention relates to a method of identifying inhibitors of epithelial-mesenchymal transition (EMT). The method as described herein may comprise: comparing different sets of image data obtained from one or more cell colonies before (T1) and after (T2) exposure to a possible inhibitor of epithelial-mesenchymal transition. The method as described herein may further comprise

measuring the cell number and a spreading coefficient value in the one or more cell colonies for determining cell count ratio (CCR) and normalized cell dispersion ratio (CDR) for the one or more colonies. Thus, a possible inhibitor may be identified to be an inhibitor of EMT if the determined CCR and CDR indicates that the possible inhibitor i) does not or marginally inhibit growth and inhibits EMT, or inhibits growth and inhibits cell dispersion and optionally inhibits also EMT, or iii) may be cytotoxic and inhibits cell dispersion and optionally inhibits also EMT. In one example, the EMT may be the process wherein epithelial cells lose their epithelial characteristics and transform to typical mesenchymal cells.

In the method as described herein, “image data” refers to images of one or more cell colonies that may be obtained by multiple adjacent field images with or without interstice in between. In particular, the image data may be obtained by at least 1, 2, 3, 4, 5, 6, 7, 8, 9, 10 or more adjacent field images without interstice in between. The image data may be obtained or provided as different sets of image data obtained from one, 2, 3, 4, 5, 6, 7, 8, 9, 10 or more cell colonies. Rather than using representative snapshots, the image data obtained for the method as described herein may encompass all cells in the cell receiving container or vessel. This eliminates the issue of sampling bias, a common problem for high-content image analysis especially when cell distribution is not uniform. In addition, when the entire cell population is analyzed, ratiometric analysis (i.e. comparing T1 and T2 images) may be employed to describe the growth of the cell colony (Cell Count Ratio/CCR) as well as to derive the colony dispersion over time (Cell Dispersion Ratio/CDR).

In one example, each set of image data may be positioned or segmented independently of the presence of nuclei. In particular, the nuclei may be positioned or segmented independently of other fields of images to prevent artifact at the field border. Nuclei positioning or segmentation as used herein may be achieved by the use of known algorithm for processing images, for example, but not limited to, wavelet transform and/or watershed algorithm. In particular, the combination of wavelet transform and watershed algorithm advantageously provides fast, accurate and robust noise and unhomogenous background analysis that is suitable for high-content screening. Furthermore, watershed algorithm allows the splitting of nuclei clusters observed in tightly formed colonies. Images from different nucleic positions or segmentations would then be combined together to provide a large image of the whole, cell colony. Nucleic positions within the cell colony may then be used to determine how much the one or more cell colonies have dispersed. The dispersion or spreading of the one or more cell colonies may then be calculated by a spreading coefficient.

Accordingly, a spreading coefficient is derived from nuclei positions within the cell colony for determining how much the one or more cell colonies have dispersed. As used herein, the “spreading coefficient” may be defined as the standard deviation of cell positions in the one or more cell colonies relative to the center of the one or more colonies. The spreading coefficient may be calculated according to the formula:

$$sp = \frac{1}{\# Col} {\sum }_{c ∈ Col} \sqrt{{( {c}_{x} - {Col}_{x} )}^{2} + {( {c}_{y} - {Col}_{y} )}^{2}}$$

where Col indicates all cells of a colony, #Col is the total cell number, [Colₓ, Col_{y}] is the average position of all nuclei in the colony, [cₓ, c_{y}] is the position of the cell c.

In one example, a cell colony of the one or more cell colonies may be defined to be a cell colony by applying morphological dilation on the nucleus segmentation of a cell body forming part of a possible cell colony. That is, cell colonies or cell bodies may be estimated by applying a morphological dilation on the nucleus segmentation or position using a disk of about 10 pixels, about 20 pixels, about 30 pixels, about 40 pixels, about 50 pixels, about 60 pixels or more pixels in diameter. In particular, the disk may be of about 30 pixels (48 μm) in diameter. In some examples, cells may be contiguous, such as in a cell colony and nuclei segmentation or position dilation will result in the formation of continuous region areas with surrounding cells, which may then be identified as colonies. As would be apparent to the skilled person, a sample well for analysis may contain one or more large colonies that may correspond to the initial cell spot and one or more smaller colonies. Smaller colonies as provided herein includes, but not limited to, single cells, small clusters of cells, dust and contaminants. In one example, smaller colonies may be excluded from further analysis. That is, only images of nuclei contained in one or more large colonies may be retained for further analysis.

In one example, cell numbers and spreading coefficient value in the one or more cell colonies may be measured to determine cell count ratio (CCR) and normalized cell dispersion ratio (CDR). CCR and CDR values may be determined by combining calculated data obtained from image data obtained from before (T1) or after (T2) exposure to a possible inhibitor.

In one example, CDR threshold may be set suitably between positive controls' average CDR and negative controls' average CDR. Exemplary CDR threshold may be about 10%, about 20%, about 30%, about 40%, about 50%, about 60%, about 70% or about 80% of positive controls' average CDR and negative controls' average CDR. In one example, the CDR threshold may be set at about 50% between positive controls' average CDR and negative controls' average CDR.

In one example, negative controls may be determined by treating cells/cell colonies with carriers or solutions used to dilute the compound of interest. Cells/cell colonies in negative control population may be untreated cells/cell colonies. A suitable carrier or solution for negative control includes DMSO and the like.

In one example, positive controls may be determined by treating cells/cell colonies with compounds known to have EMT inhibitory effect. Examples of positive controls include, but not limited to, AG1478, JNJ-38S77605 and BMS-536924.

In another example, CCR threshold may be set at a growth rate that, corresponds to normal growth rate of cells/cell colonies during the T1-T2 time period, in the absence of an EMT inducer. Exemplary CCR threshold is about 1.0, about 1.2, about 1.3, about 1.4, about 1.5, about 1.6, about 1.7, about 1.8 or about 1.9 of growth rate in the absence of an EMT inducer. In the presence of an EMT inducer, the CCR of cells/cell colonies may reach from about 2 to about 4 of growth rate. In particular, CCR threshold may be set at about 1.5 growth rate, which corresponds to normal growth rate of cells during the T1-T2 time period, in the absence of an EMT inducer.

In one example, cytotoxicity in cells/cell colonies may be determined by observing the CCR value. CCR value of less than about 1.5, about 1.4, about 1.3, about 1.2, about 1.1 or about 1.0 may correspond to cytotoxicity. In one example, CCR value of less than 1.0 may correspond to cytotoxicity as this would correlate to fewer cells in T2 than in T1. In one example, to validate cytotoxicity observed, cytotoxicity in cells/cell colonies may optionally be performed with a separate cell viability assay such as MTS assay.

The term “marginally” as used herein refers to at least less than about 40%, about 30%, about 20%, about 10%, about 9%, about 8%, about 7%, about 6%, about 5%, about 4%, about 3%, about 2% or about 1% growth inhibition. In particular, marginal growth inhibition as used herein refers to close to at least less than about 1% or barely no growth inhibition observed. The term “marginally” as used herein may be interchangeably used with “substantially no growth” or “essentially no growth”. In one example, the term “marginally inhibit growth” may refer to CCR value of between about 1.1 to about 1.9, about 1.2 to about 1.8, about 1.3 to about 1.7 or about 1.4 to about 1.6. In one example, inhibition of growth may refer to CCR value of between about 0.9 to about 1.5, 1.0 to about 1.4 or 1.1 to about 1.3.

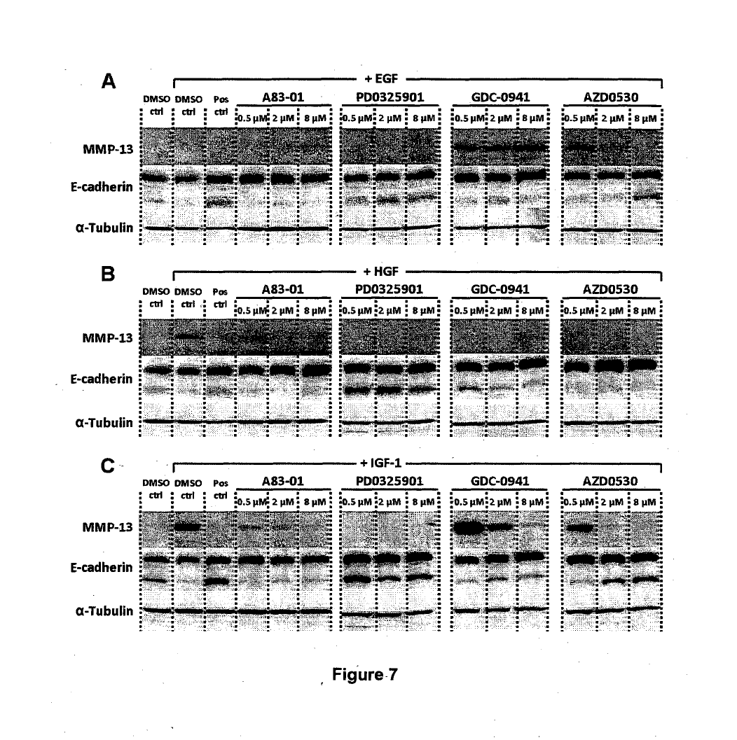

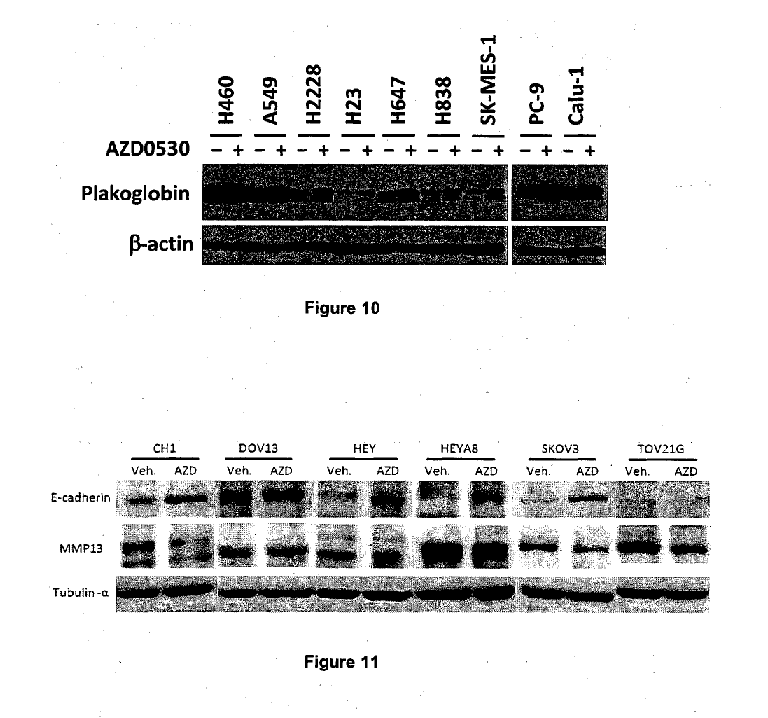

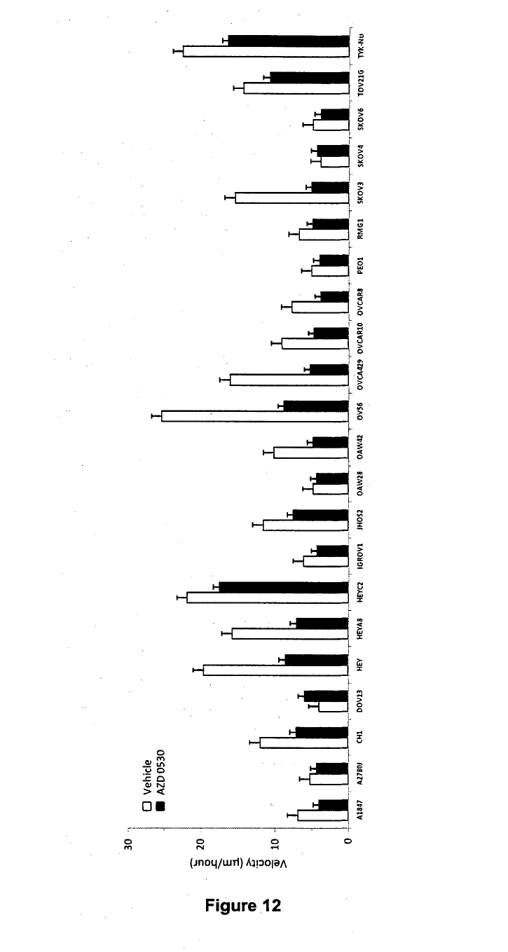

In one example, the inhibition of EMT may be measured and/or validated by assays commonly known by the person skilled in the art, for example Western Blots to detect changes in abundance of EMT-related proteins such as, but not limited to, E-cadherin, Plakoglobin, Vimentin, MMP-2, MMP-9, Snail, Slug, Twist, and the like). FIGS. 7, 10 and 11 of the present disclosure provide non-limiting examples of assays that may be performed to determine inhibition of EMT.

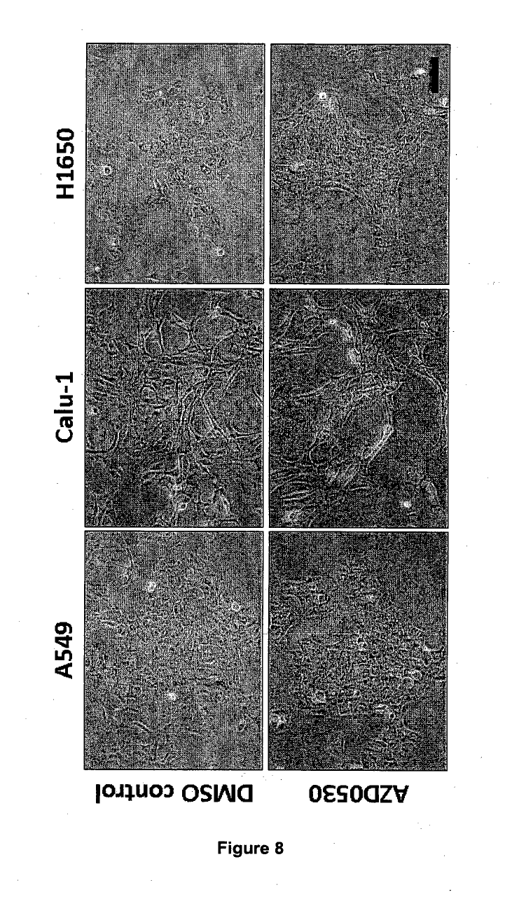

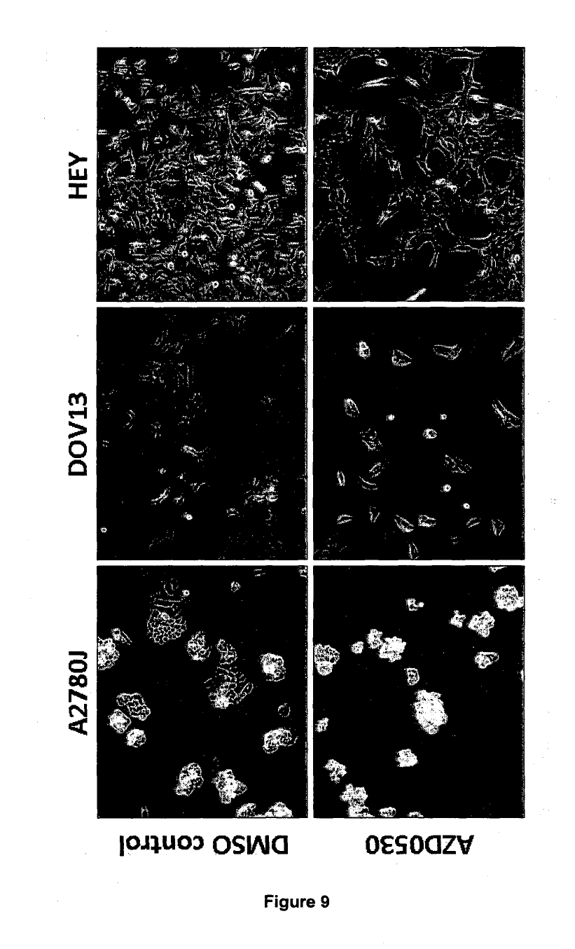

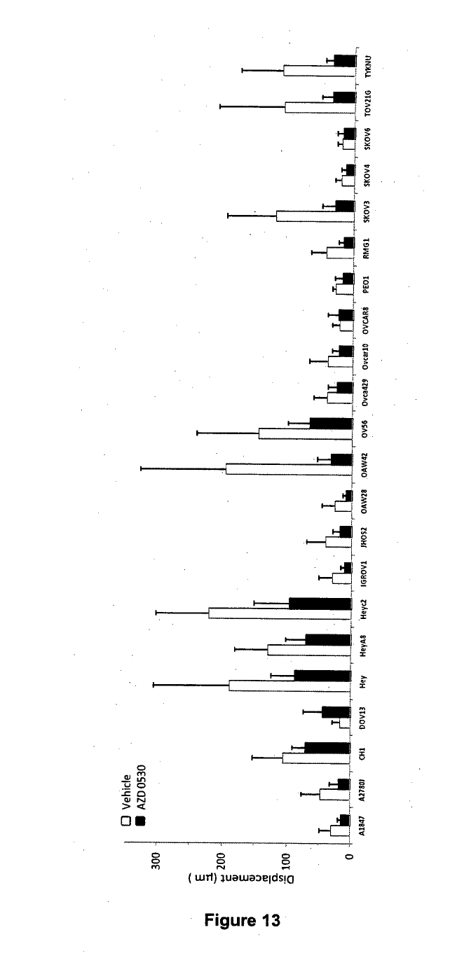

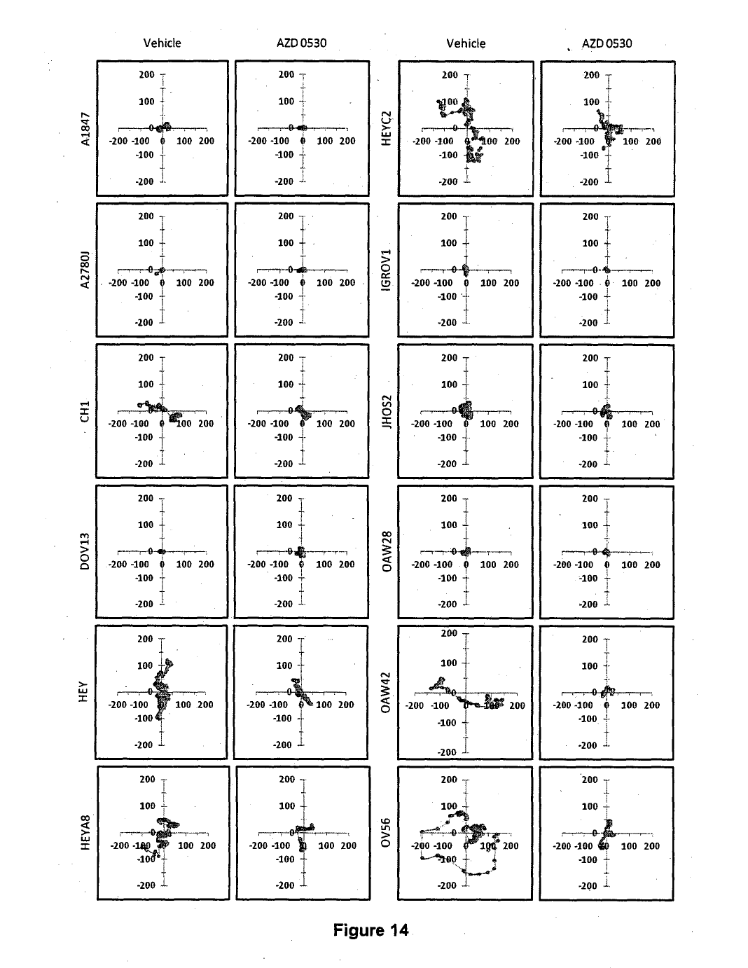

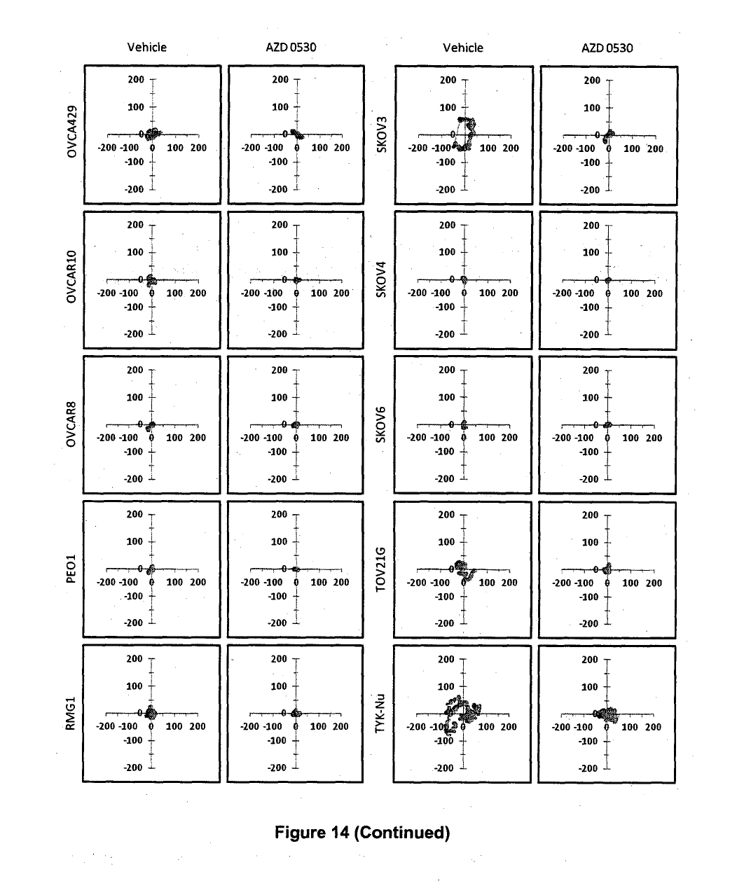

In one example, the inhibition of cell dispersion may be measured by assays commonly known by the person skilled in the art, for example cell movement tracking, cell morphology assessment, transwell invasion assays, and the like. FIGS. 8, 9, 12, 13 and 14 provide non-limiting examples of assays that may be performed to determine inhibition of cell dispersion.

The term “normalization” as used herein refers to CDR or CDR % value of each set of image data calculated by comparing the respective positive and negative controls for each induction condition as the boundary limits as follows:

$$CCR = \frac{\# {Col}_{T 2}}{\# {Col}_{T 1}} ; CDR = \frac{{sp}_{T 2}}{{sp}_{T 1}} ; CDR \% = \frac{CDR - {CDR}_{pos}}{{CDR}_{neg} - {CDR}_{pos}} \times 100 \%$$

where CDRₚₒₛ and CDR_{neg} are the average CDR values of the negative and positive control wells respectively in each test plate.

In one example, the time between obtaining the sets of image data obtained before (T1) and after (T2) the exposure to the possible inhibitor may be selected to be sufficient to allow the possible inhibitor to cause a reaction indicative for the activity of the possible inhibitor. The time between T1 and T2 may be optimized to allow for EMT and/or sufficient cell motility and/or dispersion to occur in the cell colonies without allowing significant cell proliferation response to occur as EMT quantification may be masked if the motility response is too slow. A non-limiting range of the time may be between 1 to 36 hours, or between 1 to 24 hours, or between 5 to 36 hours, or between 5 to 24 hours, or between 10 to 24 hours, or between 15 to 24 hours, or between 20 to 24 hours, about 24 hours, or the like. In one example, the method as disclosed herein may further comprise continuous recording of the one or more colony after addition of a growth factor.

In one example, the one or more cells may be: cells grown at the surface of a container until colony formation before exposure to the possible inhibitor. In one example, the cells may be provided initially as cell suspension drop deposited onto a dry surface. To provide high-throughput, accurate and consistent deposition of cells, a multi-channel liquid-handling device or apparatus known in the art may be utilized. The cells would then be confined within the drop and will be kept confined until the one or more colonies formed. The confinement of cells within the drop advantageously allows fast attachments and growth of sparsely attached cells such that colonies are formed at a short period of time. The confinement of cells also ensures ease determination of the boundary of the initial epithelial colony. To encourage effective cell attachment, the drop of cell suspension may be cultured at 37° C. and 5% CO₂ environment. In one example, the drop of cell suspension may be provided in small amount such as, but not limited to, about 0.1 μl, about 0.2 μl, about 0.3 μl, about 0.4 μl, about 0.5 μl, about 0.6 μl, about 0.7 μl, about 0.8 μl, about 0.9 μl, about 1.0 μl, about 1.5 μl, about 2.0 μl, or more. To prevent evaporation of the drop of suspension, drop of cells may be confined in sealed cell receiving container or vessel. Thus, in one example, the one or more cells may be comprised in a CO₂ independent culture medium to compensate for the lack of circulating CO₂ in the sealed container or vessel.

In one example, the one or more cells may be selected from cells that demonstrate epithelial-mesenchymal transition within its life cycle. The one or more cells may demonstrate fast epithelial-mesenchymal transition phenotypic response to known epithelial-mesenchymal transition stimuli, including, but not limited to at least one endogenous and/or at least one exogenous growth factors. The fast epithelial-mesenchymal transition advantageously allows for epithelial-mesenchymal transition quantification with minimal masking by cell proliferation response that may occur if the motility response is too slow. In one example, the epithelial-mesenchymal transition in the one or more cells may be initiated and maintained by exposure of the one or more cells to at least one exogenous and/or endogenous growth factor, for example epidermal growth factor (EGF), hepatocyte growth factor (HGF), Insulin like growth factor-1 (IGF-1), fibroblast growth factor (FGF), angiopoietin-1 (Ang1), enodostatin (Endo), interleukins-1, -4, -6, -8, platelet-derived growth factor (PDGF), vascular endothelial growth factor (VEGF), tumor necrosis factor alpha (TGFα), stromal-derived factor-1 (SDF1), transforming growth factor beta (TGFβ), or a combination thereof. In one example, the epithelial-mesenchymal transition in the one or more cells may be initiated and maintained by exposure of the one or more cells to at least one exogenous growth factor.

In particular, the one or more cells may be selected from cells which react to the possible inhibitor of epithelial-mesenchymal transition. A non-limiting example of cells as disclosed herein includes but not limited to Nara Bladder Tumor No. 2 cells (NBT-II), A549 lung adenocarcinoma line and Madin-Darby Canine Kidney (MDCK) cell lines.

In one example, the cells may be exposed to the possible inhibitor at different concentrations. For example, the cells may be exposed to the possible inhibitor at about 0.1 μM, about 0.5 μM, about 1.0 μM, about 2.0 μM, about 2.5 μM, about 3.0 μM, about 4.0 μM, about 5.0 μM, about 6.0 μM, about 7.0 μM, about 8.0 μM, about 9.0 μM, about 10.0 μM, about 20.0 μM or more. The cells may be exposed to the possible inhibitor for about 1 hour, 2, 3, 4, 5, 6, 7, 8, 9, 12, 16, 24, 30, 36, 48, 72, 96 or more hours. In one example, the growth factor may be added to the one or more cells after the addition of the possible inhibitor. In particular, the growth factor may be added to the one or more cells between 1 to 36 hours, or between 1 to 24 hours, or between 5 to 36 hours, or between 5 to 24 hours, or between 10 to 24 hours, or between 15 to 24 hours, or between 20 to 24 hours, or about 24 hours after addition of the possible inhibitor. In another example, the possible inhibitor may be added at the same time the cell colonies are formed or after the cell colonies are formed.

In one example, the possible inhibitor may be small molecule compounds.

In one example, the one or more cells as described herein may be stably or transiently transfected with a reporter gene. In one example, the reporter gene may be an optically detectable reporter gene that may be detected for imaging purposes that allows for the tracking of cells. The optically detectable reporter gene may encode for a fluorescent or luminescent protein that may be detected using any known imaging microscopy that allows live-cell imaging, for example, an epifluorescent/confocal microscope, an epifluorescent/confocal microplate imager and the likes.

In one example, the possible inhibitor may be identified to be an inhibitor of EMT if the determined CCR and normalized CDR indicate that the possible inhibitor inhibits EMT and does not or marginally inhibit growth. The inhibition of epithelial-mesenchymal transition by a possible inhibitor as identified by the method as described herein may be indicative of an anti-cancer drug or a drug that may be used in cancer treatment.

In one example, the method as described herein may further comprise conducting of downstream analysis of protein or gene expression of the cell colonies after incubation with, growth factor and inhibitor. Possible downstream assays that may be used include, but not limited to Western-Blot, Southern Blot, PCR, RT-PCR or the like. Thus, in one example, the method as described herein may further comprise a Western-Blot with cells of the one or more cell colonies after incubation with growth factors.

The method of screening of the present invention advantageously may be implemented or programmed into a robotic liquid handler to deposit consistent, reproducible cell colonies as confined spots onto multi-well plates.

For example, also disclosed is a device using the method as described herein for identifying inhibitors of epithelial-mesenchymal transition. The device may be a high-throughput screening device that comprises a multi-channel liquid-handling machine. This ensures the formation of consistent, reproducible cell colonies in well confined spots within the cell receiving container or vessel. In one example, the device may comprise a droplet dispenser unit to eject droplets of cell suspensions into a cell receiving container or vessel such as, but not limited to, a plate, well or flask. In one example, the cell receiving container may be a well. In particular, the cell receiving container may be a multi-well container, such as a 96 well plate. In one example, the container is sealable to avoid evaporation of cell culture medium after dispensing of the cell suspension into the cell receiving container.

Also disclosed is a system that may comprise a device as described herein and a camera for recording an EMT time-lapse video. In one example, the system may further comprise means to carry out downstream analysis, such as a Western blot, with cells of the one or more cell colonies.

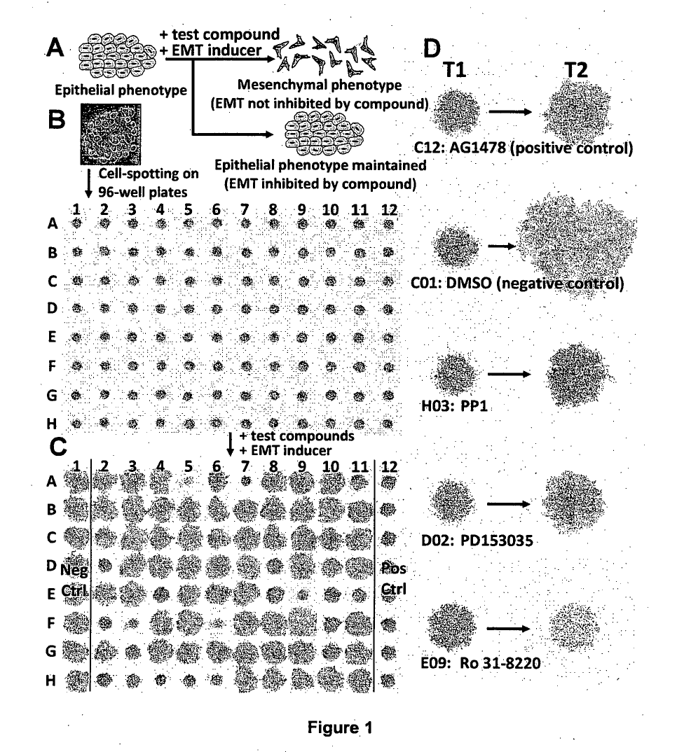

In one example, a method for identifying or screening inhibitors of epithelial-mesenchymal transition in a proliferative disease is provided. The method may be carried out as conducted in Example 1. The concept behind the method as described herein is illustrated in FIG. 1A, wherein provided are cells with epithelial phenotype, which upon suitable stimulation (i.e. EMT inducer) can transform themselves to have mesenchymal phenotype. A test compound of a putative EMT inhibitor may then be added to the cells either together, before or after the addition of EMT inducer. Cells that are added with test compounds with potential EMT inhibiting properties would maintain their epithelial phenotype (for example uniform maintenance of colonies). In contrast, if the test compound does not have any EMT inhibiting properties, these cells will not maintain their epithelial phenotype and transform to having mesenchymal phenotype (for example dispersed and absence of uniform colonies).

One possible arrangement of the method as described herein is illustrated in FIG. 1B. In this example, upon spotting of cells on 96-well plates, test compounds and EMT inducer are added to the plate. After suitable incubation period, various pattern of cell dispersion, proliferation or death would be observed (FIGS. 1C and 1D).

In one example, the method as described herein comprise the step of capturing images of cells before (T1) and after (T2) the addition of the inhibitor. FIG. 1D provides one example of how a typical pattern of cell dispersion would look like. In particular, FIG. 1D, right hand column provides for images of cells before (T1) the addition of the inhibitors and left hand column provides for images of cells after (T2) the addition of the inhibitors. At T1; the cells can be observed to form a unified colony. At T2, various cell dispersion patterns can be observed. First row of FIG. 1D illustrates a positive control pattern (i.e. cells added with known EMT inhibitor AG1478). Second row of FIG. 1D illustrates a negative control pattern (i.e. cells without any inhibitor). In this negative control, cells are clearly dispersed throughout the sample well. The third and fourth columns of FIG. 1D illustrate a pattern of inhibition by compounds PP1 and PD153035, respectively. The fifth column illustrates cells treated with growth inhibitor or toxic compound.

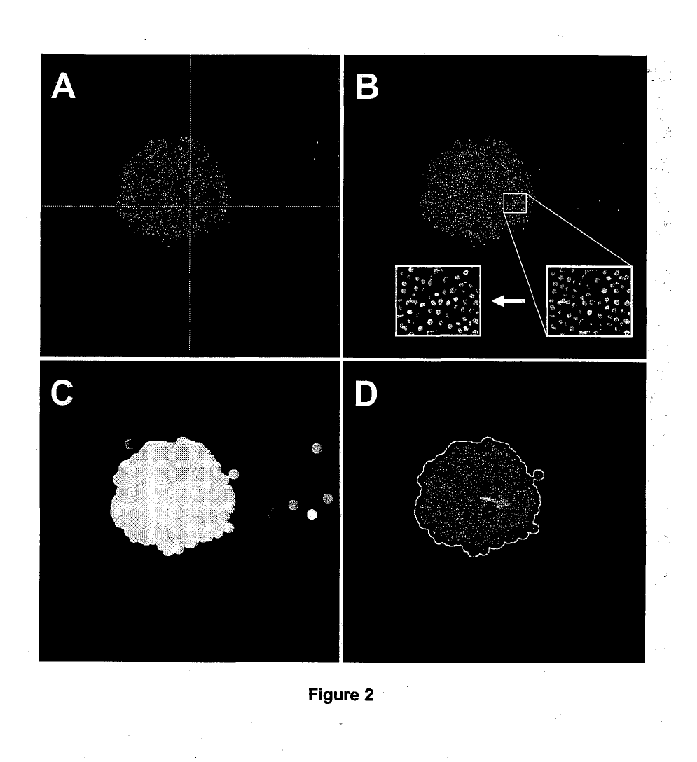

In one example, the captured images of T1 and T2 may then be analysed for any changes in cell proliferation and cell dispersion pattern as compared to a positive and negative control. Cell proliferation and cell dispersion pattern may be analysed as illustrated in Example 2. Briefly, total cell numbers in a colony and a cell dispersion or spreading coefficient value may be measured through image segmentation routine as illustrated in FIG. 2. Firstly, in this particular example, four adjacent images are stitched together (FIG. 2A). It would also be possible to make the measurement based on a single image, less than 4 images or more than 4 images. Second, nucleic positions or segmentation that may be performed using wavelet transform and watershed algorithm steps may be applied to identify all nuclei in the well (FIG. 2B). Any outlier colonies or cell clusters are also identified as this would be useful to determine cell dispersion ratio (FIG. 2C). The total cell number is then determined by the total nuclei count within the colony (FIG. 2D).

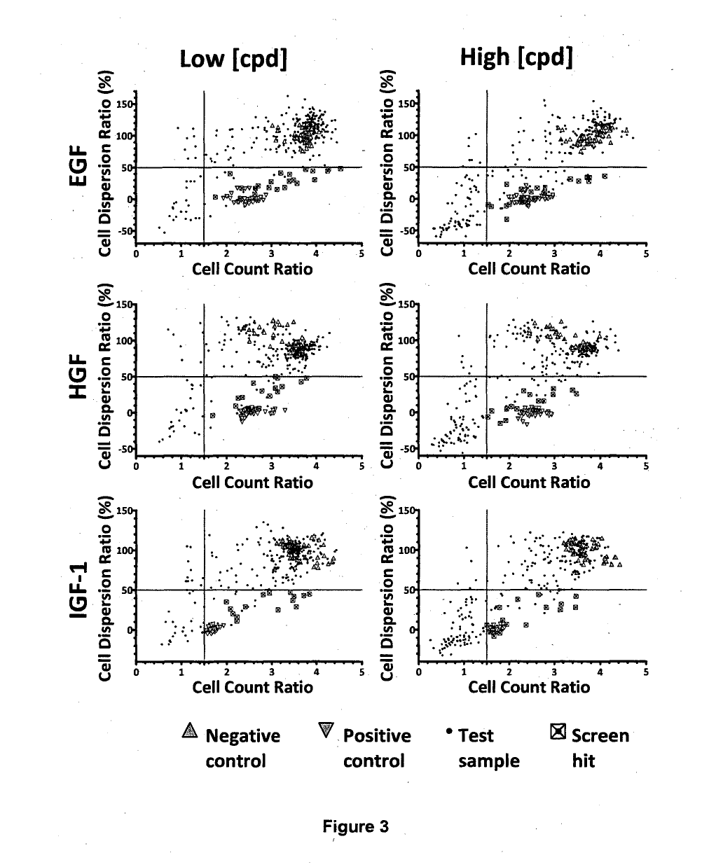

In one example, possible useful inhibitors are detected through analysis of cell dispersion ratio and cell count ratio. Any cells that displays cell dispersion ratio vs. cell count ratio above a threshold criteria would be considered to be a possible useful inhibitors (Example 3 and FIG. 3).

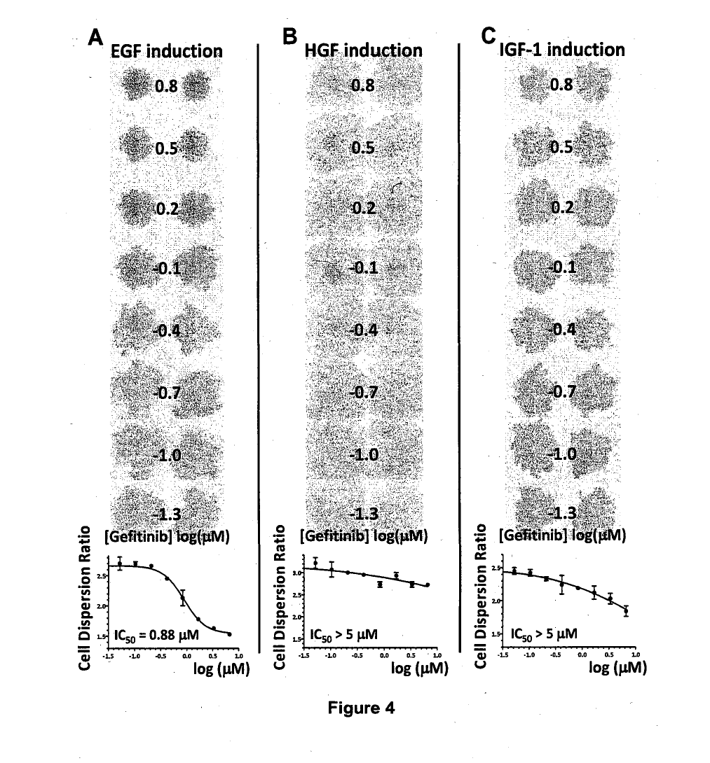

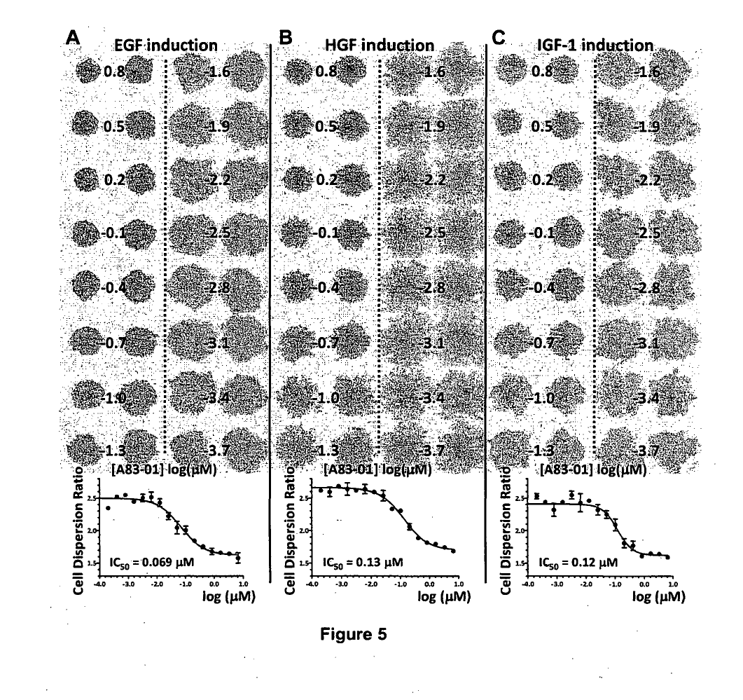

In one example, as illustrated in Example 3 and Table 2, 25 shortlisted compounds were tested for their EMT inhibitory properties. An exemplary profile of an inhibitor (for example Gefitinib) that is effective against EMT by specific growth factors only is illustrated in FIG. 4. An exemplary profile of an inhibitor (for example A83-01) that is potent against EMT with all three growth factors is illustrated in FIG. 5.

In one example, downstream assays as those described in Examples 4 and 5 may be incorporated in the method as described herein to provide for secondary validation. In particular, time-lapsed video may be used to observe cell colony behaviour after the addition of potential inhibitor. Alternatively, the expression of EMT markers such as E-cadherin and matrix metalloproteinase-13 may be used to confirm the phenotype of cells treated with potential inhibitor (FIG. 7).

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXAMPLES

Non-limiting examples of the invention will be further described in greater detail by reference to specific Examples, which should not be construed as in any way limiting the scope of the invention.

### Materials and Methods

### Preparation of Compound Stock Plates

Test compounds are purchased from various vendors (Selleck Chemicals, Sigma Aldrich, SYN|thesis MedChem, and Tocris Bioscience). Compound stocks are assembled in 96-well V-bottom plates (Greiner). For screening studies, test compounds at both 0.25 mM and 1.0 mM concentrations in DMSO were prepared, with each occupying a single well in columns 2-11 of the stock plates. For dose response studies, test compounds were prepared in duplicate wells and serially diluted in DMSO, starting with a 1.0 mM concentration. Stock plates were stored at −20° C. and thawed to room temperature-before use.

### Cell Line

Nara Bladder Tumor No. 2 (NBT-II) cells were purchased from American Type Culture Collection and were maintained for less than six months after receipt. The cell line was authenticated in Institute of Molecular and Cell Biology by desmoplakin and E-cadherin markers and by their morphology. The cells were tested Mycoplasma free prior to the experiments (MycoAlert, Lonza).

### Spot Migration Assay

NBT-II cells were stably transfected with mcherry-fluorescent H2B and maintained in DMEM supplemented with 10% fetal bovine serum (FBS, Thermo Scientific), 1 μg/ml puromycin (Sigma) and 100 units/ml penicillin-streptomycin (1× pen-strep, Invitrogen). Cells were grown to 80% confluency in tissue culture flasks prior to plating. Cells were trypsinized and concentrated to a density of 5×10⁶ cell/ml in CO₂-independent medium (Invitrogen) supplemented with 10% FBS. The cell suspension was then evenly dispersed into the wells of two columns of a 96-well V-bottom plate. Using a robotic liquid-handling station (Bravo, Agilent Technologies), 0.5 μl of cell suspension was transferred from the two columns of the cell suspension-loaded plate and deposited into the center of the wells of two columns of a 96-well clear bottom, black assay plate (Corning). This process was repeated six times so that all 96 wells of the assay plate were deposited with a cell suspension spot. The plate was then sealed to minimize evaporation of the cell suspension spots and transferred to a 37° C., 5% CO₂ incubator to allow for cells to attach to the culture surface. After 1 hour, the plate was gently washed with medium once to remove unattached cells, refreshed with 100 μl of assay medium (DMEM supplemented with 10% FBS and 1× pen-strep), and then further incubated to allow for cell-cell contacts to establish in the cell colonies.

After 4 hours of incubation, the cell colonies for each well were imaged using a confocal microplate imager (MetaXpress Ultra, Molecular Devices) with 10× Plan Fluor objective, 561 nm laser excitation and 593/40 nm emission filter configuration. Four tiled, non-overlapping images were acquired around the center of each well, and were then stitched together during image analysis to generate a montage covering an area of 3.2 mm×3.2 mm. These images (T1) represent the initial state of the cell colonies before EMT induction.

After the T1 images were acquired, 1 μl of test compounds were transferred from compound stock plates and added to the assay plates. Appropriate negative controls (1 μl DMSO) and positive controls (1 μl 1.0 mM compound in DMSO) were also added into columns 1 and 12 of each assay plate, respectively. The assay was optimized to use AG1478, JNJ-38S77605 and BMS-536924 as reference positive control compounds for EGF-, HGF- and IGF-1-induced EMT, respectively. The cultures were then further incubated overnight.

The next day, 50 μl of growth factor-containing medium was added to each well of the assay-plates. For each of the growth factor-induced EMT spot migration assays, the final growth factor concentrations in each well is optimized to be 20 ng/ml EGF (Sigma), 4 ng/ml HGF (Calbiochem) or 150 ng/ml IGF-1 (R&D Systems), respectively. The cultures were then incubated for another 24 hours, to allow for EMT and sufficient cell motility/dispersion to occur in the cell colonies.

Finally, the cell colonies were imaged again using the microplate imager, as described above. These images (T2) represent the final state of the cell colonies after compound treatment and EMT induction. The acquired T1 and T2 image sets for each assay plate were then sent for image analysis.

### Image Analysis Routine

The acquisition of each well was obtained by four adjacent field images with no interstice in between. Each field image was loaded and nuclei were initially segmented independently of other fields to prevent artifact at the field border. Nuclei segmentation was achieved by combining a wavelet transform which is robust to noise and unhomogeneous background, and a watershed algorithm based on intensities to split nuclei clusters. Mask of nuclei segmentation of the different fields were then stitched together in order to obtain a large segmentation of the whole well. Cell bodies were estimated by applying a morphological dilation on the nucleus segmentation using a disk of 30 pixels (48 μm) in diameter. In cells that are contiguous, such as in a cell colony, nuclei segmentation dilation will result in the formation of continuous region areas with surrounding cells which can then be identified as colonies. In general, a well will contain one big colony (corresponding to the initial cell spot) and several much smaller colonies (corresponding to single cells, small cluster of cells, dust and contaminations. Only nuclei contained in the biggest colony were kept and subsequently analyzed. A spreading coefficient was derived from nuclei positions in order to measure how much the colony had dispersed. The spreading coefficient is defined as the standard deviation of the cell positions in the cell colony relative to the center of the colony:

$$sp = \frac{1}{\# Col} {\sum }_{c ∈ Col} \sqrt{{( {c}_{x} - {Col}_{x} )}^{2} + {( {c}_{y} - {Col}_{y} )}^{2}}$$

where Col indicates all cells of a colony. #Col is the total cell number, [Colₓ, Col_{y}] is the average position of all nuclei in the colony, [cₓ, c_{y}] is the position of the cell c. The coefficient sp is homogenous with a distance and indicates the relative cell dispersion from the colony center.

Total cell number and spreading coefficient values for each well were then exported into an Excel sheet. The Cell Count Ratio (CCR) and Cell Dispersion Ratio (CDR) values were calculated by combining data calculated from T1 and T2 images for each well, while the normalized CDR or CDR % of each well calculated by taking the CDR values of negative and positive controls as the boundary limits:

$$CCR = \frac{\# {Col}_{T 2}}{\# {Col}_{T 1}} ; CDR = \frac{{sp}_{T 2}}{{sp}_{T 1}} ; CDR \% = \frac{CDR - {CDR}_{pos}}{{CDR}_{neg} - {CDR}_{pos}} \times 100 \%$$

where CDRₚₒₛ and CDR_{neg} are the average CDR values of the negative and positive control wells respectively in each test plate.

### EMT Time-Lapse Video

EMT inhibitory effects of selected compounds were validated by NBT-II epithelial colony time-lapse videoscopy. NBT-II cells were plated onto a 12-well plate (BD) at a low density of 2,000 cells per well in 1 ml of assay medium. Cells were allowed to grow and form epithelial colonies for a period of 72 hours. The cultures were then refreshed with assay medium containing test compounds and further incubated overnight. The next day, growth factor (EGF, HGF or IGF-1) was added prior to video imaging. Video imaging of individual cell colonies was performed using a video microscope incubator system (Axiovert-200M, Carl Zeiss). Time-lapse images were taken at 5 min intervals for 19 h.

### Western Blots

NBT-II cells were treated with compounds at 0.5, 2 and 8 μM overnight and incubated with a growth factor for 24 h. Cells were lysed with protease/phosphatase inhibitor-containing RIPA buffer. Proteins are separated in 8% polyacrylamide gels and transferred to PDVF membranes. Membranes were blocked in 5% BSA and incubated at 4° C. overnight with MMP-13 (Millipore), E-cadherin (BD) and α-tubulin (Sigma) primary antibodies. Membranes were then developed with HRP-conjugated secondary antibody (Amersham) and ECL substrate (Millipore).

For western blot experiments with other cell lines, cells are treated either with AZD0530, such as 2 μM AZD0530 or DMSO, such as 0.02% DMSO control for 48 h. The samples are then processed using the same procedure as described above. The samples were probed with MMP-13, E-cadherin, plakoglobin (Cell Signaling), α-tubulin, and β-actin (Cell Signaling) antibodies.

### Cell Movement Tracking Time-Lapse Videoscopy

Cells were maintained in RMPI supplemented with 10% fetal bovine serum and 1× pen-strep. Cells were grown to 80% confluency in tissue culture flasks prior to plating. Cells were trysinized and plated onto a 12-well plate (BD) at a low density of 2000 cells per well in 1 ml of RMPI. Each ovarian cell line was seeded into two wells of a 12 well tissue culture plate. For each ovarian cell line, one of the two wells was treated with 2 μM AZD0530 and the other well treated with 0.02% DMSO control for 48 hours. Cells were imaged using a time-lapse video microscope incubator system (Leica) controlled by Metamorph software (Molecular Devices). Time-lapse images were taken at 5 min intervals for 24 hours and saved as tiff format. Each individual tiff images were compiled into stack images using the stack creation function in Metamorph. Each cell was tracked using the particle tracking function in Metamorph software. The tracking function detected and recorded the x and y coordinates of the cell being tracked for each image in the stack. The x and y coordinates were used to plot the track of the individual cell in excel. The total distance travelled, displacement and average velocity of the tracked cell were then tabulated.

### Statistical Analysis

Error bars in CDR dose response plots represent standard deviation of replicate samples. CDR IC₅₀ values were calculated through sigmoidal curve fitting of CDR dose response plots using GraphPad Prism software.

### Example 1

### EMT Spot Migration Assay Design and Assembly

An overview of the EMT screening assay is illustrated in FIG. 1. To screen for compounds that have the propensity to inhibit EMT in cells induced by exogenous growth factor signaling (FIG. 1A), a high-throughput method of forming compact, consistent epithelial cell colonies in 96 wells was designed. Using a multi-channel liquid-handling machine, a 0.5 μl high density suspension of cells is directly deposited into the center of wells of 96-well plate, in a highly consistent manner (FIG. 1B). As the cell suspension drop is initially deposited onto a dry surface, the cells are confined within the drop, and will be kept confined until they firmly attach. Thus, the drop area essentially determines the boundary of the initial epithelial colony that will be formed in the well. As the environmental conditions for effective cell attachment are 37° C. and 5% CO2, the plate is sealed to prevent the 0.5 μl drop from evaporating in the incubator. In addition, CO₂ independent medium is used instead of normal culture media to compensate for the lack of circulating CO₂ in the sealed plate. This sealed environment can be maintained for more than an hour for cell attachment to be completed, after which the wells can be gently washed and refreshed with normal culture media. Due to the initial compactness, the attached cells can quickly establish cell-cell contacts and consistent epithelial colonies will form hours after cell plating.

In this study, the NBT-II reporter cell line was used. The cells were stably transfected with H2B-mcherry to label the nuclei, so that the migration of these cells could be tracked through live-cell fluorescent imaging. NBT-II is an ideal cell model for the study of EMT because of its fast EMT phenotypic response to several known EMT stimuli, such as EGF, HGF and IGF-1. As evident in FIG. 1C, a complete EMT cell motility response was achieved within 24 hours after the addition of an EMT stimulus. This short response time is important for the screening assay of this example, as the EMT quantification may be masked by cell proliferation response if the motility response is too slow.

For compound screening, the spot migration assay to identify compounds that could inhibit EMT induced by EGF, HGF. or IGF-1 signaling was optimised. Appropriate compounds (AG1478, JNJ-38877605 and BMS-536924) were selected as reference positive controls for each EGF, HGF and IGF-1 EMT assay, respectively. The screen is conceptualized as a high-content imaging assay, whereby colony nuclei in each well are imaged and analyzed prior to compound treatment (T1 images), and 24 hours after EMT induction (T2 images). The effect of the screening compounds in this assay was grouped into three categories: [1] compounds that are cytotoxic or growth inhibitory to cells: [2] compounds that can inhibit EMT and are not growth inhibitory to cells; and [3]compounds that are not EMT or growth inhibitory. The cell colony examples shown in FIG. 1D highlight these three different categories. The grouping of compounds into these three categories was determined through image analysis of the plate images.

### Example 2

### Image Analysis and Assay Robustness

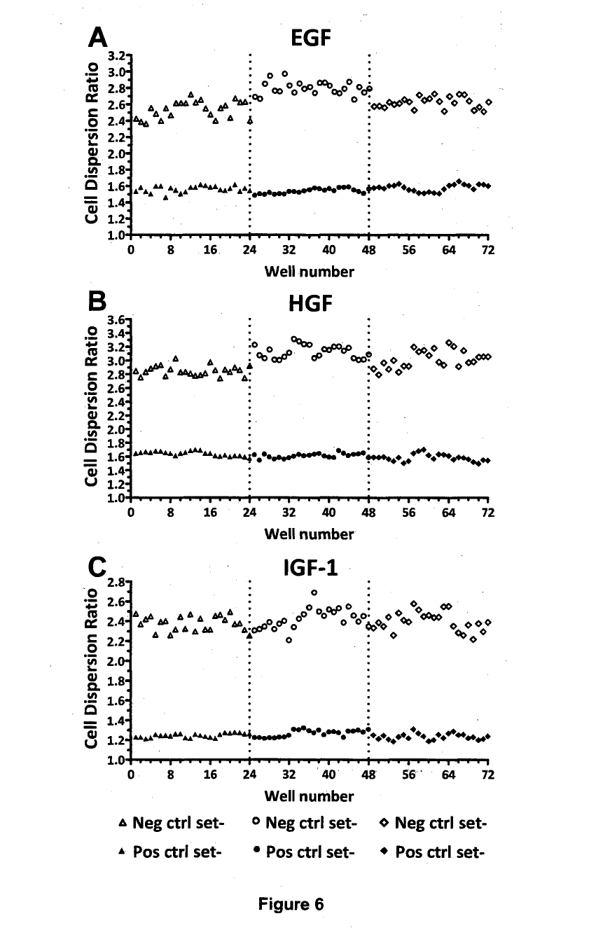

The analysis routine developed for this screening assay, is illustrated in FIG. 2. For each well image, total cell numbers in the colony and a spreading coefficient value were measured through the image segmentation routine. The spreading coefficient is defined as the standard deviation of the cell positions in the colony relative to the center of the colony. By combining time-course images of T1 and T2, the derived measurements Cell Count Ratio (CCR) and Cell Dispersion Ratio (CDR) was obtained, which correspond to the cell growth status and the cell migration/scattering status of each cell colony, respectively. The results generated from these two ratio parameters were used to assess the EMT inhibitory properties of the test compounds. The uniformity and robustness of the assay (positive and negative control well CDR uniformity plots shown in FIG. 6) was also assessed. The validated CDR signal was robust in the screening assay, where intra-plate Z-factor was consistently above 0.5 between positive and negative control signals.

### Example 3

### Identification of Potential EMT Inhibitors in Targeted Compounds Screen

A collection of 267 targeted inhibitor compounds was tested to assess if any of them could inhibit EGF-, HGF- or IGF-1-induced EMT in this screening assay (FIG. 3). The complete data for the EMT targeted inhibitors screen is listed in Table 1 below. For hit finding, compounds that are not growth inhibitory (CCR≧1.5) and could inhibit cell dispersion (CDR %≦50%) with a wide concentration range more than 0.5 log differences were identified. Compounds were therefore tested at two concentrations (6.67 μM and 1.67 μM) in the EMT screen. Based on the screening data generated and the selection criteria that were set, 25 compounds that may potentially inhibit EGF-, HGF- or IGF-1-induced EMT were shortlisted.

To assess the EMT inhibition potency of the 25 shortlisted compounds, the compounds were retested at diluting concentrations starting from 6.67 μM, using the same EMT spot migration assay for EGF, HGF or IGF-1 signaling. CDR dose response plots were generated for each compound/growth factor combination and the CDR IC₅₀ values corresponding to the EMT inhibition potency were determined. Compounds that were effective against EMT by specific growth factors only were identified (FIG. 4). Compounds that were potent against EMT with all three growth factors were also identified (FIG. 5). A summary of the CDR IC₅₀ values for the 25 compounds is listed in Table 2. In general, these compounds are grouped by their primary signaling molecules that they are designed to target. This grouping strategy facilitates the validation of this assay, where a c-Met inhibitor (e.g. PF-04217903) specifically inhibited HGF-induced EMT, while an EGFR inhibitor (e.g. Gefitinib) only inhibited EGF-induced EMT. Four groups of compounds were also identified for targeting ALK5, MEK, PI3K and SRC that were inhibitory to several EMT-inducing growth factors.

TABLE 1
EMT spot migration data for 267 compounds tested at 1.67 and 6.67 μM concentrations, under EGF-, HGF- or IGF-1-induced EMT conditions.

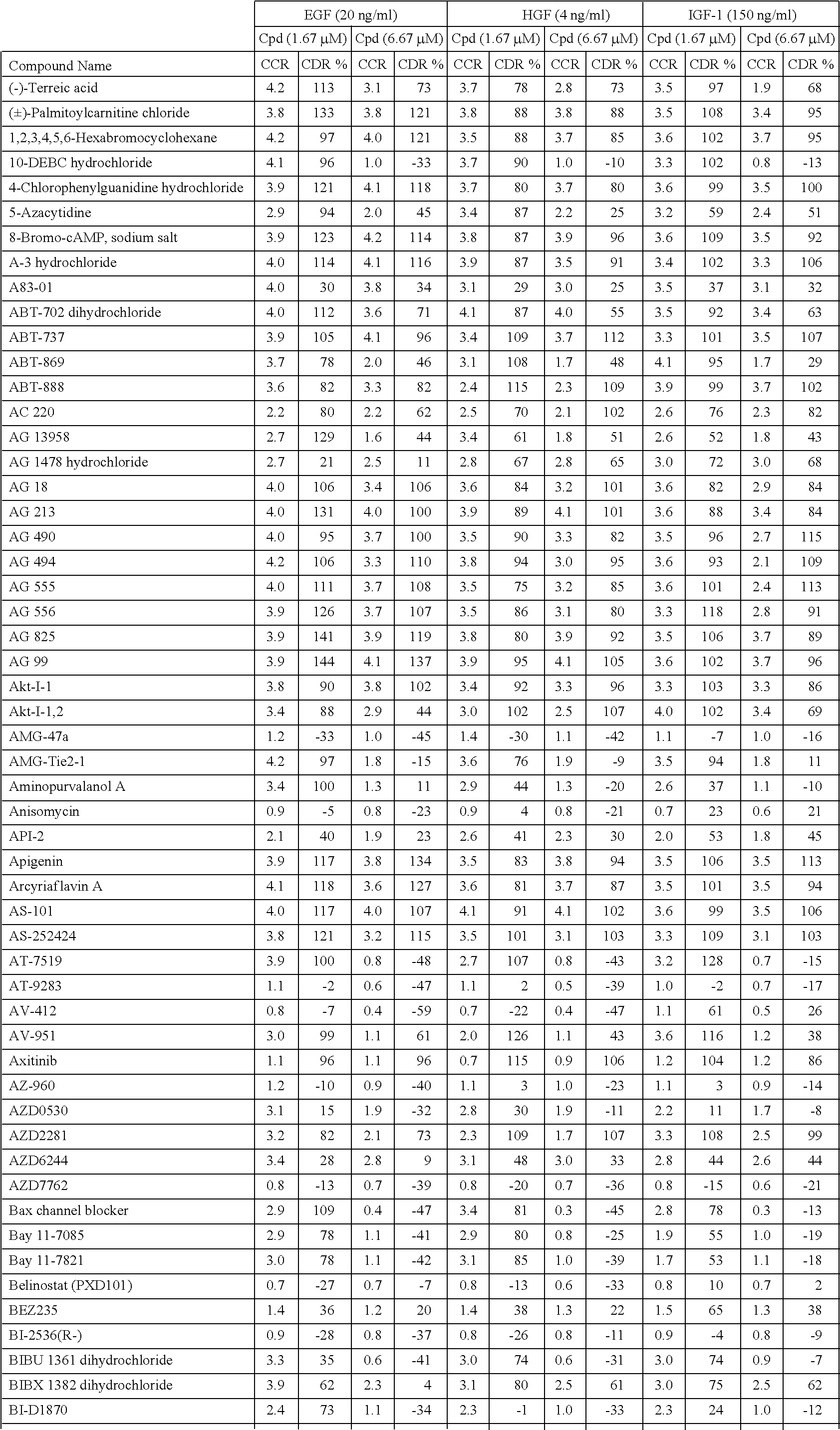

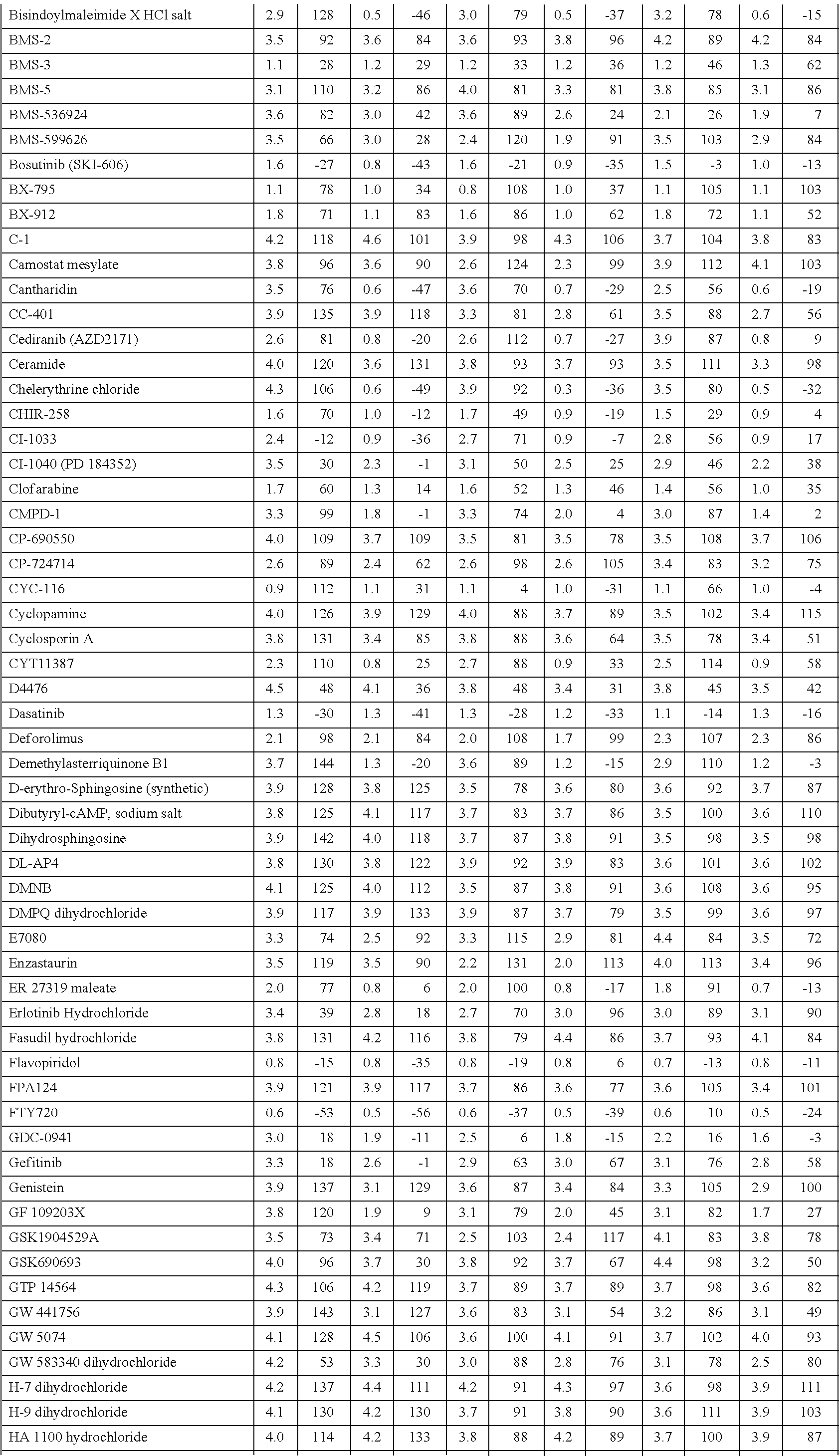

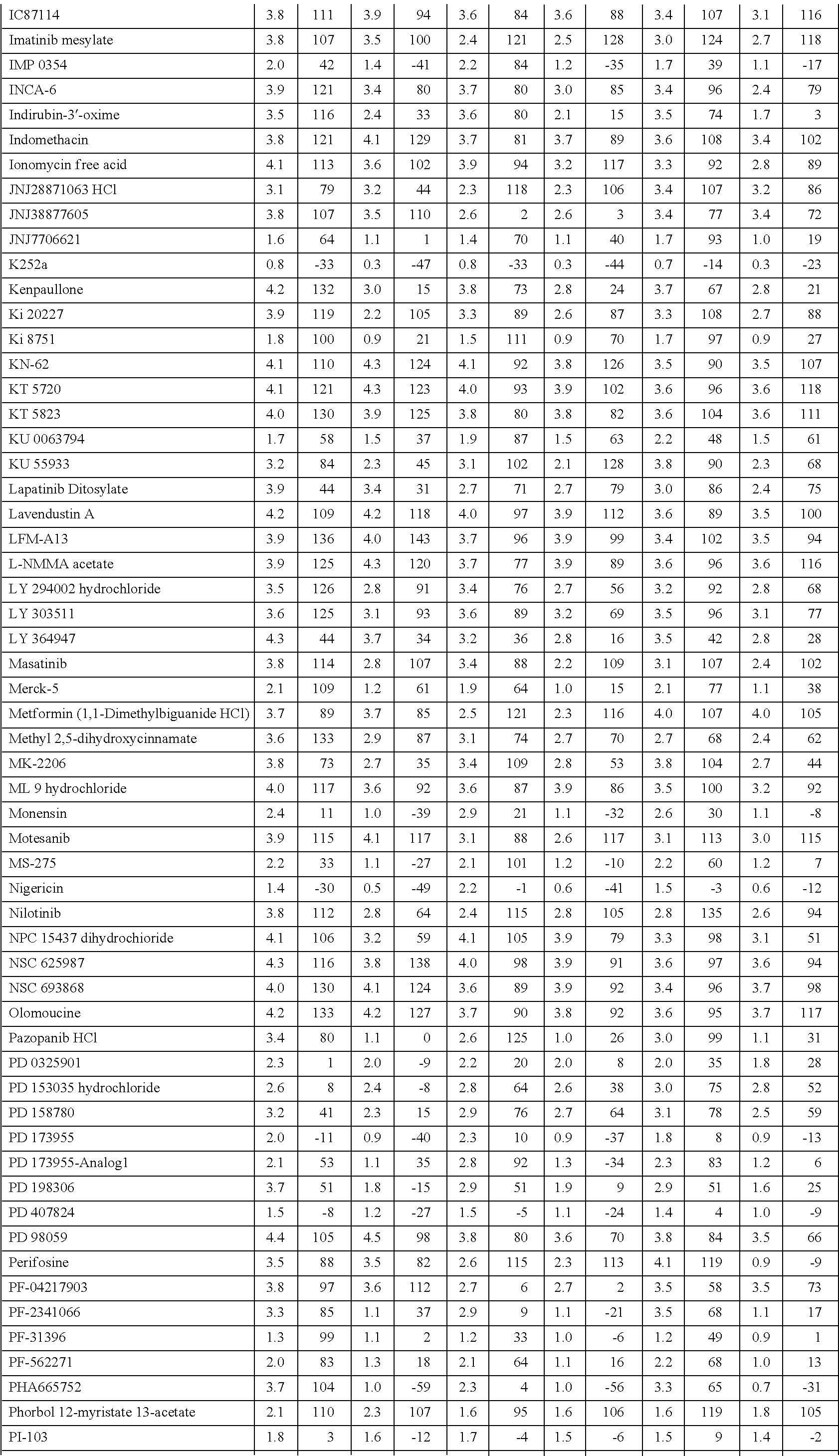

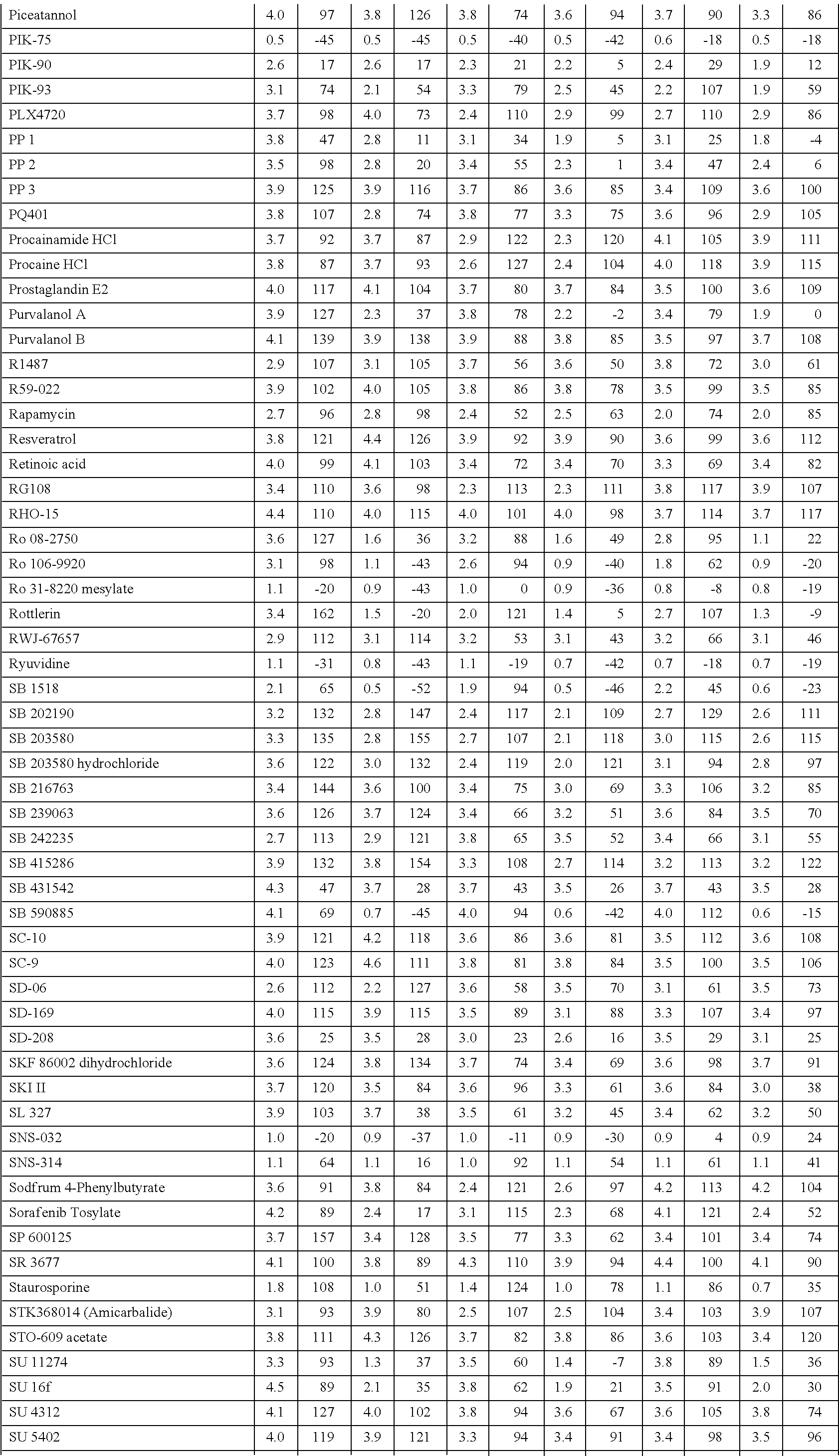

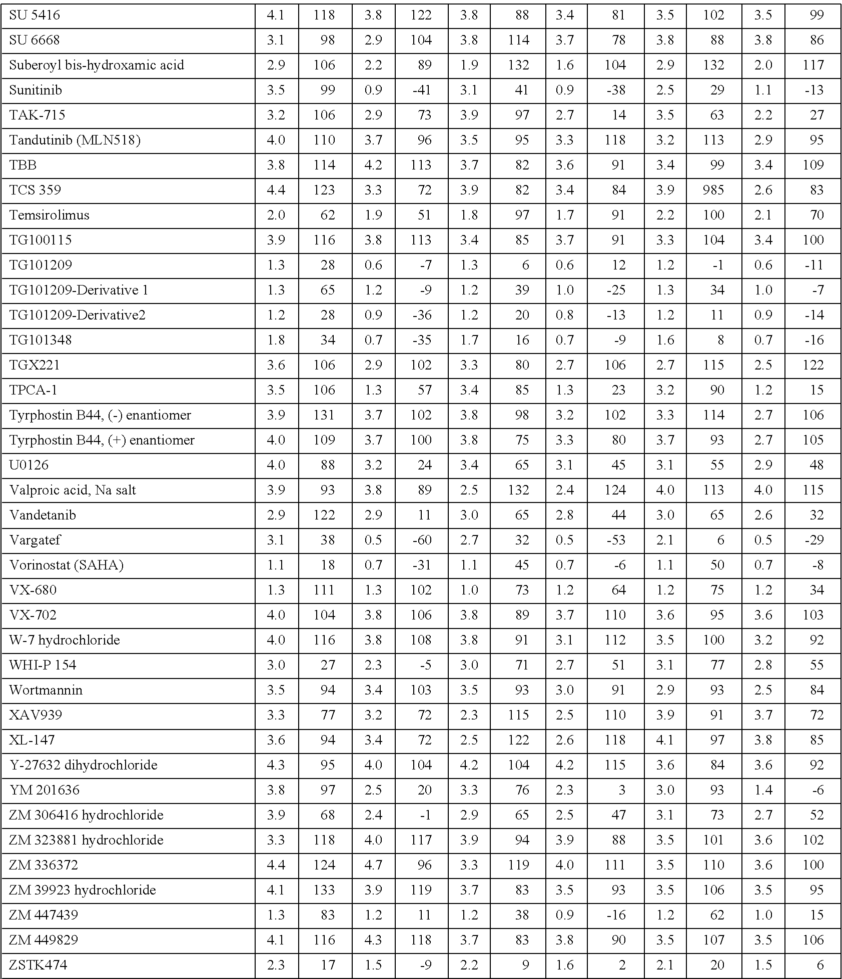

Data where CCR ≦ 1.5 are highlighted dark gray, indicating compound condition was growth inhibitory.
Data where CDR % < 50% are highlighted in light gray, indicating compound condition was dispersion inhibitory.

TABLE 2
Summary of EMT inhibitor CDR IC₅₀ values
against EGF-, HGF- or IGF-1-induced EMT.

| | | Cell Dispersion | | | |
|---|---|---|---|---|---|
| | | IC ₅₀ (nM) | | | Development |
| Name | Target | EGF | HGF | IGF-1 | stage^{a} |
| JNJ-38877605 | c-MET | >5000 | 94 | >5000 | Phase 1 |
| PF-04217903 | c-MET | >5000 | 55 | >5000 | Phase 1 |
| AG1478 | EGFR | 330 | >5000 | >5000 | Research |
| Erlotinib | EGFR | 950 | >5000 | >5000 | FDA |
| | | | | | approved |
| Gefitinib | EGFR | 880 | >5000 | >5000 | FDA |
| | | | | | approved |
| Lapatinib | EGFR | 620 | >5000 | >5000 | FDA |
| | | | | | approved |
| PD153035 | EGFR | 550 | >5000 | >5000 | Research |
| PD158780 | EGFR | 1200 | >5000 | >5000 | Research |
| WHI-P154 | EGFR | 800 | >5000 | >5000 | Research |
| BMS-536924 | IGF-1R, IR | >5000 | 2300 | 170 | Research |
| A83-01 | ALK5 | 69 | 130 | 120 | Research |
| D4476 | ALK5 | 1100 | 1400 | 1900 | Research |
| LY-364947 | ALK5 | 140 | 180 | 240 | Research |
| SB-431542 | ALK5 | 1600 | 940 | 820 | Research |
| SD-208 | ALK5 | 85 | 110 | 150 | Research |
| AZD6244 | MEK1/2 | 840 | 790 | 880 | Phase 1-2 |
| CI-1040 | MEK1/2 | 1000 | 820 | 1200 | Phase 2 |
| PD0325901 | MEK1/2 | 31 | 20 | 8.9 | Phase 1-2 |
| GDC-0941 | PI3K | 740 | 380 | 500 | Phase 1 |
| PI-103 | PI3K | 680 | 380 | 400 | Research |
| PIK-90 | PI3K | 950 | 400 | 620 | Research |
| ZSTK474 | PI3K | 850 | 410 | 660 | Phase 1 |
| API -2 | PKB, AKT | 1300 | >5000 | >5000 | Phase 1 |
| AZD0530 | SRC, ABL | 560 | 510 | 240 | Phase 1-2 |
| PP1 | SRC | 2300 | 2000 | 1200 | Research |

^{a}Clinical trials information: ClinicalTrials.gov

### Example 4

### Secondary Assays to Validate EMT Inhibitors

One compound was selected from each target group and their EMT inhibitory response were validated via time lapse video. The videos confirmed that an EGFR, c-Met and IGF-1R inhibitor could specifically inhibit EGF-, HGF- or IGF-1-induced EMT, respectively, as expected (data not shown). ALK5, MEK, PI3K and SRC targeting compounds were also shown to inhibit EMT induced by all three growth factors (data not shown). This finding is surprising as these compounds are not the immediate and direct antagonists of the growth, factors linked to EMT signaling.

The possibilities that ALK5, MEK, PI3K and SRC targeting compounds could modulate the expression of EMT markers, such as E-cadherin and matrix metalloproteinase-13 (MMP-13) under EMT-activated conditions were also investigated (FIG. 7). With the exception of P13K inhibitor GDC-0941, the compounds in general abrogated MMP-13 expression in growth factor-treated samples, PI3K inhibition has been previously shown to augment MMP-13 expression. PD0325901 and AZD0530 was also shown herein to augment E-cadherin expression in all three growth factor-treated conditions, while A83-01 and GDC-0941 restored the E-cadherin protein levels. These results demonstrate that selective inhibition of ALK5, MEK and SRC could block EMT by restoring E-cadherin-mediated cell adhesion and reducing the invasion promoting MMP-13 and motility. These findings are consistent with previous reports showing that ALK5, MEK and SRC play a role in cell motility and tumor progression, while PI3K predominately regulates cell proliferation.

### Example 5

### Synergism Effects of Combinations

The synergism effects between compound combinations in inhibiting the EMT phenotype was also investigated (Table 3). In this assay, EMT inhibition combination index (CI) values of ALK5 inhibitor A83-01 and c-Met inhibitor JNJ-38877605 combination against HGF-induced EMT was investigated. Cell dispersion ratio dose response profiles of A83-01 and JNJ-38877605 at fixed combinations ratios of 1:4, 1:2, 1:1 and 3:1 were generated using the spot migration assay. To determine if the EMT inhibitory effects obtained with different compound combinations were synergistic, the inhibition effect CI values were calculated according to the Chou0Talalay method using CalcuSyn software (Biosoft) (where CI>1.1, antagonism; CI=0.9-1.1, additive effect, CI=0.2-0.9, synergism; and CI<0.2 strong synergism). The results indicated that the combination treatment acted synergistically against HGF-induced EMT.

TABLE 3
EMT inhibition index (CI) values of ALK5 inhibitor
A83-01 and c-Met inhibitor JNJ-38877605 combination
against HGF-induced EMT.

| A83-01:JNJ-38877605 | | |
|---|---|---|
| combination ratio | 50% CI | 75% CI |
| 1:4 | 0.63 | 0.41 |
| 1:2 | 0.67 | 0.34 |
| 1:1 | 0.47 | 0.23 |
| 3:1 | 0.33 | 0.13 |

The development and implementation of an EMT inhibition screening assay adapted for high-throughput, high-content screening of small molecule compounds is described herein. In one example, a robotic liquid handler was programmed to deposit consistent, reproducible cell colonies as confined spots onto multi-well plates (FIG. 1B). This method of confining cells to generate cell colonies within a few hours has not been previously attempted (the common alternative method is to allow sparsely attached cells to grow and form colonies, and this typically takes more than 3 days).

For image analysis, the wavelet transform and watershed segmentation methods were used because the resultant nuclei segmentation is fast and accurate, suitable for high-content screening (FIG. 2). Rather than using representative snapshots of the well, in one example, the image acquisition and analysis method were designed to encompass all cells in the well (FIG. 1). This eliminates the issue of sampling bias, a common problem for high-content image analysis especially when cell distribution is not uniform. As this method accounts for all cells in the well, and the cell population per well is large (typically more than 1,000 cells), the analysis describing cell dispersion would be reliable. In addition, because the entire cell population was analyzed, ratiometric analysis (i.e. comparing T1 and T2 images) may be employed to describe the growth of the cell colony (Cell Count Ratio) as well as to derive the colony dispersion over time (Cell Dispersion Ratio). The CDR values for the plate controls, which determine the upper and lower CDR boundaries, were shown to be consistent and robust (FIG. 6), and this increases the confidence of the assay in hit determination.

The EMT spot migration assay has key advantages over traditionally described cell migration quantification methods, such as the Boyden chamber or the in vitro scratch techniques. In general, these techniques are prone to sampling bias because, for practicality reasons, only representative microscope views and not the entire well image are chosen for analysis. Another key strength of the spot migration assay is that cell proliferation and cell dispersion within each well may be quantitated simultaneously (FIG. 2). This is important it enables the selection of compounds that are predominately anti-migratory or EMT inhibitory against those that are generally toxic to cells (FIG. 3), as cell toxicity will inherently hinder cell motility. Previous attempts to screen for cell motility modulating agents are mainly based on the in vitro scratch assay, which involves creating a scratch in a confluent cell monolayer and measuring the speed at which the cell layer grows or migrates to close this “wound”. Although the method has also been adapted for high-throughput screening by using robotic-driven-pins to generate scratches on multi-well plates, it has failed to quantitatively differentiate whether the impairment of wound closure by the lest agent is due to the inhibition of cell motility or the inhibition of, cell growth pressure at the scratch front.

The assay of the present invention may be used to analyze whether targeted compounds previously selected and optimized to kill oncogene-addicted cells, can also be used to effectively inhibit EMT signaling.

The screening assay of the present invention may address the relative propensity and potency for small molecule compounds to block growth factor-induced EMT signaling. Through the primary screen and subsequent secondary assays, the druggable targets ALK5, MEK, SRC and to some extent PI3K were discovered by the method, of the present invention to play a more significant role in EMT modulation and cancer progression, as their associated targeted compounds are inhibitory to several EMT-inducing growth factors (Table 2). As would be appreciated by the person skilled in the art, the targeted compound library provided herein represents only a small subset of the targeted compounds that have been developed by universities and the pharmaceutical industry, and does not encompass all the druggable targets identified to date. Therefore, further extension of this EMT spot migration assay to include other diverse targeted compound libraries, such as the one assembled by Bamborough et al., may allow the discovery other potent EMT inhibitors and EMT modulating targets. The selected compounds may then be evaluated for their ability to revert the mesenchymal-like phenotype of cancer cells in vitro and immuno-compromised mice. The synergism effects between compound combinations in inhibiting the EMT phenotype were also investigated and disclosed herein (example in Table 3). The method of the present invention may facilitate in the design of new therapeutic modalities based on the EMT concept to interfere with tumor progression and to suppress resistance to chemotherapeutic agents.

### APPLICATIONS

The present invention may be used to screen for inhibitors of the EMT in a proliferative disease such as cancer.

Advantageously, the method of screening is robust and time efficient. The method of the present invention provides for a high-throughput and high-content screening method of screening small molecule compounds.

Advantageously, the present invention can be used to screen compound libraries for small molecule compounds that are effective in inhibiting EMT signaling in response to growth factor treatment. Thus, allowing the discovery of unknown potent EMT inhibitors and EMT modulating targets.

Advantageously, the present invention allows for simultaneous analysis of both cell growth and cell migration responses on the same test sample. The present invention also facilitates for dose titration studies to further characterize the EMT inhibition potency of screened compounds.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of identifying inhibitors of epithelial-mesenchymal transition (EMT), wherein the method comprises:
a. comparing different sets of image data obtained from one or more cell colonies before (T1) and after (T2) exposure to a possible inhibitor of epithelial-mesenchymal transition;
b. measuring the cell number and a spreading coefficient value in the one or more cell colonies for determining cell count ratio (CCR) and normalized cell dispersion ratio (CDR) for the one or more cell colonies;
wherein a possible inhibitor is identified to be an inhibitor of EMT if the determined CCR and CDR indicates that the possible inhibitor i) does not or marginally inhibit growth and inhibits EMT, or ii) inhibits growth and inhibits cell dispersion and optionally inhibits also EMT, or iii) is cytotoxic and inhibits cell dispersion and optionally inhibits also EMT.

2. The method of claim 1, wherein a possible inhibitor is identified to be an inhibitor of EMT if the determined CCR and normalized CDR indicates that the possible inhibitor inhibits EMT and does not or marginally inhibit growth.

3. The method of claim 1, wherein the spreading coefficient is derived from nuclei positions within the cell colony for determining how much the one or more cell colonies have dispersed.

4. The method of claim 1, wherein the spreading coefficient is defined as the standard deviation of cell positions in the one or more cell colonies relative to the center of the one or more colonies.

5. The method of claim 4, wherein the spreading coefficient is calculated according to the following formula:
$$sp = \frac{1}{\# Col} {\sum }_{c ∈ Col} \sqrt{{( {c}_{x} - {Col}_{x} )}^{2} + {( {c}_{y} - {Col}_{y} )}^{2}}$$
where Col indicates all cells of a colony, #Col is the total cell number, [Colₓ, Col_{y}] is the average position of all nuclei in the colony, [cₓ, c_{y}] is the position of the cell c.

6. The method of claim 1, wherein time between obtaining the sets of image data obtained before (T1) and after (T2) the exposure to the possible inhibitor is selected to be sufficient to allow the possible inhibitor to cause a reaction indicative for the activity of the possible inhibitor.

7. The method of claim 6, wherein the time between obtaining the sets of image data obtained before (T1) and after (T2) the exposure to the possible inhibitor is between 1 to 36 hours, or between 1 to 24 hours, or between 5 to 36 hours, or between 5 to 24 hours, or between 10 to 24 hours, or between 15 to 24 hours, or between 20 to 24 hours, or about 24 hours.

8. The method of claim 1, wherein the one or more cells are cells grown at the surface of a container until colony formation before exposure to the possible inhibitor.

9. The method of claim 8, wherein the one or more cells are comprised in a CO₂-independent culture medium.

10. The method of claim 1, wherein the possible inhibitor is added at the same time the cell colonies are formed or after the cell colonies are formed.

11. The method of claim 1, wherein the one or more cells are selected from cells which react to the possible inhibitor of epithelial-mesenchymal transition.

12. The method of claim 11, wherein the one or more cells are selected from the group consisting of Nara Bladder Tumor No. 2 cells (NBT-II), A549 lung adenocarcinoma line and Madin-Darby Canine Kidney (MDCK) cell lines.

13. The method of claim 1, wherein the one or more cells are transfected with a detectable reporter gene.

14. The method of claim 13, wherein the report gene is an optically detectable reporter gene.

15. The method of claim 14, wherein the optically detectable reporter gene is a encoding for a fluorescent or luminescent protein.

16. The method of claim 1, wherein the image data are obtained using an epifluorescent/confocal microscope or an epifluorescent/confocal microplate imager.

17. The method of claim 1, wherein epithelial-mesenchymal transition in the one or more cells is initiated and maintained by exposure of the one or more cells to at least one (exogenous) growth factor.

18. The method of claim 17, wherein the at least one growth factor is selected from the group consisting of EGF, HGF and IGF-1.

19. The method of claim 1, wherein inhibition of epithelial-mesenchymal transition by a possible inhibitor is indicative of an anti-cancer drug or a drug that can be used in cancer treatment.

20. The method of claim 1, wherein the one or more cells are exposed to the possible inhibitor at different concentrations.

21. The method of claim 1, wherein the at least one growth factor is added to the one or more cells after addition of the possible inhibitor.

22. The method of claim 21, wherein the at least one growth factor is added to the one or more cells between 1 to 36 hours, or between 1 to 24 hours, or between 5 to 36 hours, or between 5 to 24 hours, or between 10 to 24 hours, or between 15 to 24 hours, or between 20 to 24 hours, or about 24 hours after addition of the possible inhibitor.

23. The method of claim 1, wherein the method further comprises continuous recording of the one or more cell colony after addition of a growth factor.

24. The method of claim 1, wherein the method further comprises conducting of a Western-Blot with cells of the one or more cell colonies after incubation with growth factors.

25. The method of any one of claim 1, wherein a cell colony of the one or more cell colonies is defined to be a cell colony by applying morphological dilation on the nucleus segmentation of a cell body forming part of a possible cell colony.

26. A device using the method referred to in claim 1 for identifying inhibitors of epithelial-mesenchymal transition.

27. The device of claim 26, wherein the device is a high-throughput screening device.

28. The device of claim 26 comprising a multi-channel liquid-handling machine.

29. The device of claim 26 comprising a droplet dispenser unit to eject droplets of cell suspension into a cell receiving container.

30. The device of claim 29, wherein the cell receiving container is a well.

31. The device of claim 29, wherein the cell receiving container is a multi-well container, such as a 96 well plate.

32. The device of any one of claim 29, wherein the container is sealable to avoid evaporation of cell culture medium after dispensing of the cell suspension into the cell receiving container.

33. A system comprising a device according to claim 26 and a camera for recording an EMT time-lapse video.

34. The system of claim 33 further comprising means to carry out a Western blot with cells of the one or more cell colonies.
